(19) United States
(12) Patent Application Publication
(10) Pub. No.: US 2024/0327338 A1
(43) Pub. Date: Oct. 3, 2024

(54) ORGANIC SALT, RESIST COMPOSITION INCLUDING THE SAME, AND METHOD OF FORMING PATTERN USING THE SAME

(71) Applicant: Samsung Electronics Co., Ltd., Suwon-si (KR)

(72) Inventors: Aram JEON, Suwon-si (KR); Hana KIM, Suwon-si (KR); Beomseok KIM, Suwon-si (KR); Hoyoon PARK, Suwon-si (KR); Kyuhyun IM, Suwon-si (KR); Jinwon JEON, Suwon-si (KR); Sungwon CHOI, Suwon-si (KR)

(73) Assignee: Samsung Electronics Co., Ltd., Suwon-si (KR)

(21) Appl. No.: 18/483,895

(22) Filed: Oct. 10, 2023

(30) Foreign Application Priority Data
Mar. 24, 2023 (KR) 10-2023-0039209
Apr. 14, 2023 (KR) 10-2023-0049580

(51) Int. Cl. C07C 309/58; C07C 309/42; G03F 7/004; G03F 7/029; G03F 7/20

(52) U.S. Cl. CPC C07C 309/58; C07C 309/42; G03F 7/0045; G03F 7/0048; G03F 7/029; G03F 7/2004

(57) ABSTRACT

Provided are an organic salt represented by Formula 1, a resist composition including the same, and a method of forming a pattern by using the same:

A₁₁_{⁺}B₁₁_{^{−}} Formula 1
wherein, in Formula 1,
A₁₁_{⁺} is represented by Formula 1A, and B₁₁_{^{−}} is represented by Formula 1B,

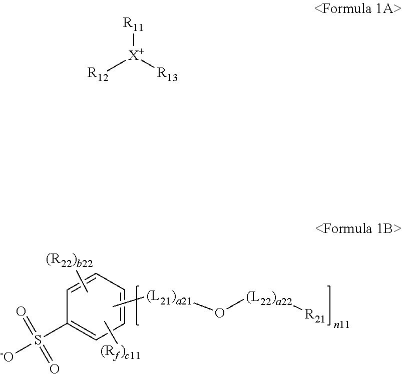

wherein descriptions of R₁₁ to R₁₃, L₂₁, L₂₂, a21, a22, R₂₁, R₂₂, R_{f}, b22, c11 and n11 in Formulae 1A and 1B are provided herein.

### CROSS-REFERENCE TO RELATED APPLICATION

This application is based on and claims priority under 35 U.S.C. § 119 to Korean Patent Application No. 10-2023-0039209, filed on Mar. 24, 2023, and Korean Patent Application No. 10-2023-0049580, filed on Apr. 14, 2023, in the Korean Intellectual Property Office, the disclosures of each of which are incorporated by reference herein in their entirety.

### BACKGROUND

### 1. Field

The disclosure relates to an organic salt, a resist composition including the same, and a method of forming a pattern using the same.

### 2. Description of the Related Art

In manufacturing semiconductors, resists that undergo changes in physical properties in response to light are used to form a micropattern. Among these resists, a chemically amplified resist is widely used. In a chemically amplified resist, an acid formed through reaction between light and a photoacid generator reacts with a base resin again to change the solubility of the base resin in a developer, thereby enabling patterning.

In particular, when high-energy rays having relatively very high energy, such as EUV rays, are used, there may be a drawback in that the number of photons may be remarkably small even when irradiated with light having the same energy. Therefore, there may be a need for a photoacid generator capable of acting effectively even when used in a small amount, and capable of providing improved sensitivity and/or resolution.

### SUMMARY

Provided are an organic salt capable of acting as a photoacid generator that can provide improved sensitivity and/or resolution, a resist composition including the same, and a method of forming a pattern using the same.

Additional aspects will be set forth in part in the description which follows and, in part, will be apparent from the description, or may be learned by practice of the presented embodiments of the disclosure.

According to an embodiment of the disclosure, an organic salt represented by Formula 1 is provided.

A₁₁_{⁺}+B₁₁_{^{−}} Formula 1

In Formula 1, A₁₁_{⁺} may be represented by Formula 1A, and B₁₁_{^{−}} may be represented by Formula 1B,

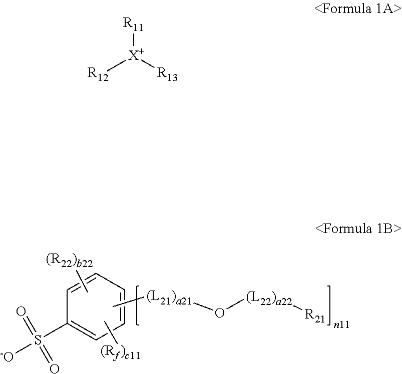

In Formulae 1A and 1B,
X may be sulfur (S), selenium (Se), or tellurium (Te),
R₁₁ to R₁₃ may each independently be a linear, branched, or cyclic C₁-C₃₀ monovalent hydrocarbon group optionally containing a heteroatom,
at least one of R₁₁ to R₁₃ may contain at least one iodine (I),
adjacent two of R₁₁ to R₁₃ may optionally be bonded to each other to form a condensed ring,
L₂₁ and L₂₂ may each independently be a single bond or a divalent linking group,
a21 and a22 may each independently be an integer from 1 to 3,
R₂₁ may be a C₁-C₃₀ cycloalkyl group optionally containing deuterium, a halogen, a hydroxyl group, a cyano group, a carbonyl group, a carboxyl group, an ether bond, an ester bond, a sulfonate ester bond, a carbonate, a lactone ring, a sultone ring, a carboxylic anhydride moiety, a haloalkyl moiety, or any combination thereof,
R₂₂ may be hydrogen; deuterium; or a halogen; or a linear, branched, or cyclic C₁-C₃₀ monovalent hydrocarbon group optionally containing a heteroatom;
R_{f} may be F or a C₁-C₃₀ alkyl group substituted with F,
b22 may be an integer from 0 to 3,
c11 may be an integer from 1 to 4, and
n11 may be an integer from 1 to 4.

According to an example embodiment, a resist composition may include the organic salt as described above, an organic solvent, and a base resin.

According to an example embodiment, a method of forming a pattern may include forming a resist film by applying the resist composition as described above, exposing at least a portion of the resist film to high-energy rays, and developing the exposed resist film by using a developer.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern, according to an embodiment;

FIG. 2A to 2C are a flowchart illustrating a method for forming a pattern, according to an embodiment.

FIGS. 3A to 3E are side cross-sectional views illustrating a method of forming a patterned structure according to an embodiment; and

FIGS. 4A to 4E are side cross-sectional views illustrating a method of forming a semiconductor device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

As the disclosure allows for various changes and numerous embodiments, particular embodiments will be illustrated in the drawings and described in detail in the detailed description. However, this is not intended to limit the disclosure to particular modes of practice, and it is to be appreciated that all changes, equivalents, and substitutes that fall within the spirit and technical scope of the disclosure are encompassed in the disclosure. In the description of the disclosure, certain detailed descriptions of related art are omitted when it is deemed that they may unnecessarily obscure the essence of the disclosure.

Although terms such as “first,” “second,” and “third” may be used to describe various components, the above terms are used only to distinguish one component from another, and the order, types, and the like of such components should not be limited by the above terms.

In the present specification, when a portion of a layer, a film, a region, a plate, or the like is referred to as being “on” or “above” another portion, it includes not only a case in which the portion is directly above or below, or on the left or right side while being in contact with the other portion, but also a case in which the portion is above or below, or on the left or right side while not being in contact with the other portion.

An expression in the singular includes an expression in the plural unless the content clearly indicates otherwise. It should be understood that, unless otherwise clearly contradicted by context, terms, such as “include” and “have,” are used to indicate the presence of stated features, numbers, steps, operations, elements, parts, components, materials, or a combination thereof without excluding in advance the possibility of the presence or addition of one or more other features, numbers, steps, operations, elements, parts, components, materials, or a combination thereof.

Whenever a range of values is recited, that range includes all values falling within that range as if explicitly stated, and further includes the boundaries of the range. Accordingly, the range “X to Y” includes all values between X and Y, including X and Y.

The term “Cₓ-C_{y}” as used herein means that the number of carbon atoms constituting a substituent is in a range of x to y. For example, the term “C₁-C₆” means that the number of carbon atoms constituting a substituent is in a range of 1 to 6, and the term “C₆-C₂₀” means that the number of carbon atoms constituting a substituent is in a range of 6 to 20.

The term “monovalent hydrocarbon group” as used herein refers to a monovalent residue derived from an organic compound containing carbon and hydrogen or a derivative thereof, and examples thereof may include: linear or branched alkyl groups (e.g., a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); monovalent saturated cycloaliphatic hydrocarbon groups (cycloalkyl groups) (e.g., a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group); monovalent unsaturated aliphatic hydrocarbon groups (an alkenyl group and an alkynyl group) (e.g., an allyl group); monovalent unsaturated cycloaliphatic hydrocarbon groups (cycloalkenyl groups) (e.g., 3-cyclohexenyl); aryl groups (e.g., a phenyl group, a 1-naphthyl group, and a 2-naphthyl group); arylalkyl groups (e.g., a benzyl group and a diphenylmethyl group); heteroatom-containing monovalent hydrocarbon groups (e.g., a tetrahydrofuranyl group, a methoxymethyl group, an ethoxymethyl group, a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group); or any combination thereof. In addition, in these groups, some hydrogen atoms may be substituted with a moiety containing a heteroatom, e.g., oxygen, sulfur, nitrogen, or a halogen atom, or some carbon atoms may be substituted with a moiety containing a heteroatom, e.g., oxygen, sulfur, or nitrogen. Thus, these groups may contain a hydroxyl group, a cyano group, a carbonyl group, a carboxyl group, an ether bond, an ester bond, a sulfonate ester bond, a carbonate, a lactone ring, a sultone ring, a carboxylic anhydride moiety, or a haloalkyl moiety.

The term “divalent hydrocarbon group” as used herein is a divalent residue and means that any one hydrogen of the monovalent hydrocarbon group is substituted with a binding site to a neighboring atom. Non-limiting examples of the divalent hydrocarbon group may include a linear or branched alkylene group, a cycloalkylene group, an alkenylene group, an alkynylene group, a cycloalkylene group, an arylene group, and those in which some carbon atoms are substituted with heteroatoms.

The term “alkyl group” as used herein refers to a linear or branched saturated aliphatic monovalent hydrocarbon group, and non-limiting examples thereof may include a methyl group, an ethyl group, a propyl group, an isobutyl group, a sec-butyl group, a tert-butyl group, a pentyl group, an iso-amyl group, and a hexyl group. The term “alkylene group” as used herein refers to a linear or branched saturated aliphatic divalent hydrocarbon group, and non-limiting examples thereof may include a methylene group, an ethylene group, a propylene group, a butylene group, and an isobutylene group.

The term “halogenated alkyl group” as used herein refers to a group in which at least one substituent of the alkyl group is substituted with a halogen, and non-limiting examples thereof may include CF₃. In this regard, the halogen may be fluorine (F), chlorine (CI), bromine (Br), or iodine (I).

The term “alkoxy group” as used herein refers to a monovalent group having the formula —OA₁₀₁ wherein A₁₀₁ may be an alkyl group. Non-limiting examples of the alkoxy group may include a methoxy group, an ethoxy group, and an isopropyloxy group.

The term “alkylthio group” as used herein refers to a monovalent group having the formula —SA₁₀₁ wherein A₁₀₁ may be an alkyl group.

The term “halogenated alkoxy group” as used herein refers to a group in which at least one hydrogen of the alkoxy group is substituted with a halogen, and non-limiting examples thereof may include —OCF₃.

The term “halogenated alkylthio group” as used herein refers to a group in which at least one hydrogen of the alkylthio group is substituted with a halogen, and non-limiting examples thereof may include —SCF₃.

The term “cycloalkyl group” as used herein refers to a cyclic saturated monovalent hydrocarbon group, and examples thereof may include monocyclic groups such as a cyclopropyl group, a cyclobutyl group, a cyclopentyl group, a cyclohexyl group, and a cycloheptyl group, and polycyclic condensed groups such as a norbornyl group and an adamantyl group. The term “cycloalkylene group” as used herein refers to a cyclic saturated divalent hydrocarbon group, and non-limiting examples thereof may include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, and a dicyclohexylmethylene group.

The term “cycloalkoxy group” refers to a monovalent group having the formula —OA₁₀₂ wherein A₁₀₂ may be a cycloalkyl group. Non-limiting examples thereof may include a cyclopropoxy group and a cyclobutoxy group.

The term “cycloalkylthio group” as used herein refers to a monovalent group having the formula —SA₁₀₂ wherein A₁₀₂ may be a cycloalkyl group.

The term “heterocycloalkyl group” as used herein may mean that some carbon atoms of the cycloalkyl group may be substituted with a moiety containing a hetero atom, such as oxygen, sulfur, or nitrogen, and the heterocycloalkyl group may contain, for example, an ether bond, an ester bond, a sulfonate ester bond, a carbonate, a lactone ring, a sultone ring, or a carboxylic anhydride moiety. The term “heterocycloalkylene group” as used herein may be a group in which some carbon atoms of the cycloalkylene group are substituted with a moiety containing a hetero atom, such as oxygen, sulfur, or nitrogen.

The term “heterocycloalkoxy group” as used herein refers to a monovalent group having the formula —OA₁₀₃ wherein A₁₀₃ may be a heterocycloalkyl group.

The term “alkenyl group” as used herein refers to a linear or branched unsaturated aliphatic monovalent hydrocarbon group containing at least one carbon-carbon double bond. The term “alkenylene group” as used herein refers to a linear or branched unsaturated aliphatic divalent hydrocarbon group containing at least one carbon-carbon double bond.

The term “cycloalkenyl group” as used herein refers to a cyclic unsaturated monovalent hydrocarbon group containing at least one carbon-carbon double bond. The term “cycloalkenylene group” as used herein refers to a cyclic unsaturated divalent hydrocarbon group containing at least one carbon-carbon double bond.

The term “heterocycloalkenyl group” as used herein may be a group in which some carbon atoms of the cycloalkenylene group are substituted with a moiety containing a hetero atom, e.g., oxygen, sulfur, or nitrogen. The term “heterocycloalkenylene group” as used herein may be a group in which some carbon atoms of the cycloalkenylene group are substituted with a moiety containing a heteroatom, e.g., oxygen, sulfur, or nitrogen.

The term “alkynyl group” as used herein refers to a linear or branched unsaturated aliphatic monovalent hydrocarbon group containing at least one carbon-carbon triple bond.

The term “aryl group” as used herein refers to a monovalent group having a carbocyclic aromatic system, and non-limiting examples thereof may include a phenyl group, a naphthyl group, anthracenyl group, a phenanthrenyl group, a pyrenyl group, and a chrysenyl group. The term “arylene group” as used herein refers to a divalent group having a carbocyclic aromatic system.

The term “heteroaryl group” as used herein refers to a monovalent group having a heterocyclic aromatic system, and non-limiting examples thereof may include a pyridinyl group, a pyrimidinyl group, and a pyrazinyl group. The term “heteroarylene group” as used herein refers to a divalent group having a heterocyclic aromatic system.

The term “cycloalkyl group” as used herein refers to a monovalent, divalent, or trivalent group in which any hydrogen of the cycloalkyl group can optionally be further substituted with a binding site.

The term “heterocycloalkyl group” as used herein refers to a monovalent, divalent, or trivalent group in which any hydrogen of the heterocycloalkyl group can optionally be further substituted with a binding site.

The term “cycloalkenyl group” as used herein refers to a monovalent, divalent, or trivalent group in which any hydrogen of the cycloalkenyl group can optionally be further substituted with a binding site.

The term “heterocycloalkenyl group” as used herein refers to a monovalent, divalent, or trivalent group in which any hydrogen of the heterocycloalkenyl group can optionally be further substituted with a binding site.

The term “aryl group” as used herein refers to a monovalent, divalent, or trivalent group in which any hydrogen of the aryl group can optionally be further substituted with a binding site.

The term “heteroaryl group” as used herein refers to a monovalent, divalent, or trivalent group in which any hydrogen of the heteroaryl group can optionally be further substituted with a binding site.

The “substituent” as used herein may be: deuterium, halogen, a hydroxyl group, a cyano group, a nitro group, a carbonyl group, a carboxylic acid group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, or a C₁-C₂₀ heteroarylthio group; a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, and a C₁-C₂₀ heteroarylthio group, each substituted with deuterium, a halogen, a hydroxyl group, a cyano group, a nitro group, a carbonyl group, a carboxylic acid group, an amino group, an ether moiety, an ester moiety, a sulfonate ester moiety, a carbonate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, or any combination thereof; and any combination thereof.

Hereinafter, embodiments of the disclosure will be described in detail with reference to the accompanying drawings, and in description with reference to the drawings, like reference numerals denote substantially like or corresponding elements, and redundant description thereof will be omitted herein. Thicknesses of various layers and regions in the drawings are exaggerated for clear illustration. In addition, in the drawings, thicknesses of some layers and regions are exaggerated for convenience of explanation. Meanwhile, embodiments set forth herein are provided for illustrative purposes only, and various modifications can be made to these embodiments.

### [Organic Salt]

An organic salt according to embodiments is represented by Formula 1 below.

A₁₁_{⁺}B₁₁_{^{−}} Formula 1

In Formula 1,
A₁₁_{⁺} may be represented by Formula 1A, and B₁₁_{^{−}} may be represented by Formula 1B;

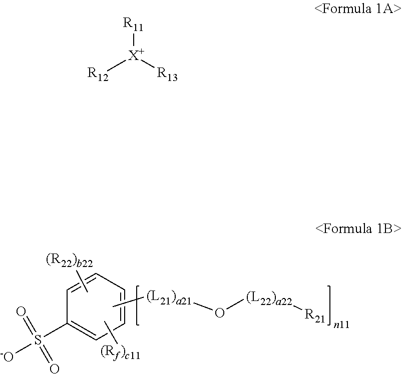

In Formulae 1A and 1B,
X may be sulfur (S), selenium (Se), or tellurium (Te);
R₁₁ to R₁₃ may each independently be a linear, branched, or cyclic C₁-C₃₀ monovalent hydrocarbon group optionally containing a hetero atom;
at least one of R₁₁ to R₁₃ may contain at least one iodine (1);
adjacent two of R₁₁ to R₁₃ may optionally be bonded to each other to form a condensed ring;
L₂₁ and L₂₂ may each independently be a single bond or a divalent linking group;
a21 and a22 may each independently be an integer from 1 to 3;
R₂₁ may be a C₁-C₃₀ cycloalkyl group optionally containing deuterium, a halogen, a hydroxyl group, a cyano group, a carbonyl group, a carboxyl group, an ether bond, an ester bond, a sulfonate ester bond, a carbonate, a lactone ring, a sultone ring, a carboxylic anhydride moiety, a haloalkyl moiety, or any combination thereof;
R₂₂ may be hydrogen; deuterium; or a halogen; or a linear; branched; or cyclic C₁-C₃₀ monovalent hydrocarbon group optionally containing a heteroatom;
R_{f} may be F, or a C₁-C₃₀ alkyl group substituted with F;
b22 may be an integer from 0 to 3;
c11 may be an integer from 1 to 4; and
n11 may be an integer from 1 to 4.

For example, in Formula 1A, X may be S.

For example, in Formula 1A, R₁₁ to R₁₃ may each independently be selected from a C₁-C₂₀ alkyl group, a C₃-C₂₀ cycloalkyl group, and a C₆-C₂₀ aryl group, each unsubstituted or substituted with deuterium, a halogen, a cyano group, a hydroxyl group, a carboxyl group, an ester moiety, a sulfonate ester moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In one or more embodiments, in Formula 1A, R₁₁ to R₁₃ may each independently be selected from a C₁-C₂₀ alkyl group, a C₃-C₂₀ cycloalkyl group, and a C₆-C₂₀ aryl group, each unsubstituted or substituted with a halogen, a cyano group, a hydroxyl group, an ester moiety, a sulfonate ester moiety, a lactone moiety, a sultone moiety, a carboxylic anhydride moiety, a methyl group, an ethyl group, a propyl group, an isopropyl group, a butyl group, an isobutyl group, a sec-butyl group, a tert-butyl group, a halogenated methyl group, a halogenated ethyl group, a methoxy group, an ethoxy group, a phenyl group, or any combination thereof.

In one or more embodiments, in Formula 1A, at least one of R₁₁ to R₁₃ may be a C₆-C₂₀ aryl group substituted with at least one I atom.

In an embodiment, in Formula 1A,
R₁₁, R₁₂, or R₁₃ may contain at least one I atom,
R₁₁ and R₁₂ may each contain at least one I, and R₁₃ may not contain an I atom, or
R₁₁ to R₁₃ may each contain at least one I.

In other embodiments, in Formula 1A, R₁₁, R₁₂, or R₁₃ may contain one I atom or two I atoms,
R₁₁ and R₁₂ may each contain one I or two I atoms, and R₁₃ may not contain I, or
R₁₁ to R₁₃ may each contain one I atom or two I atoms.

In one or more embodiments, in Formula 1, A₁₁_{⁺} may contain one I atom, two I atoms, or three I atoms.

In an embodiment, in Formula 1, A₁₁_{⁺} may be represented by Formula 1A-1:

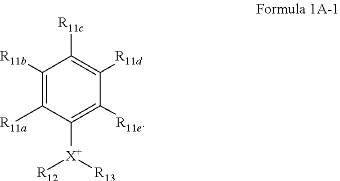

In Formula 1A-1,
X may be S, Se, or Te,
R₁₁ₐ to R₁₁ₑ may each independently be: hydrogen; a halogen; or a linear, branched, or cyclic C₁-C₂₀ monovalent hydrocarbon group optionally containing a heteroatom,
R₁₂ and R₁₃ may each independently be a linear, branched, or cyclic C₁-C₃₀ monovalent hydrocarbon group optionally containing a heteroatom,
an adjacent two of R₁₁ₐ to R₁₁ₑ, R₁₂, and R₁₃ may optionally be bonded to each other to form a condensed ring, and
at least one of R₁₁ₐ to R₁₁ₑ, R₁₂, and R₁₃ may contain at least one I atom.

In one or more embodiments, in Formula 1A-1, at least one of R₁₁ₐ to R₁₁ₑ may be I.

In one or more embodiments, in Formula 1A-1, R₁₁ₐ, R_{11b}, or R_{11c} may be I.

In one or more embodiments, in Formula 1A-1, R_{11b} and R_{11c} may each be I, or R_{11b} and R_{11d} may each be I.

In an embodiment, in Formula 1, A₁₁_{⁺} may be represented by Formula 1A-11 or 1A-12:

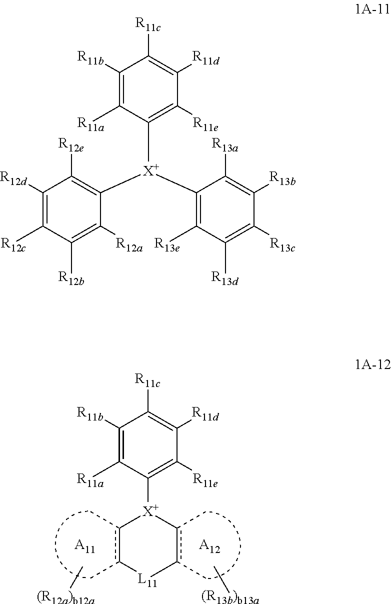

In Formulae 1A-11 and 1A-12,
X may be S, Se, or Te;
R₁₁ₐ to R₁₁ₑ, R₁₂ₐ to R₁₂ₑ, and R₁₃ₐ to R₁₃ₑ may each independently be: hydrogen; a halogen; or a linear, branched, or cyclic C₁-C₂₀ monovalent hydrocarbon group optionally containing a heteroatom;
at least one of R₁₁ₐ to R₁₁ₑ, R₁₂ₐ to R₁₂ₑ, and R₁₃ₐ to R₁₃ₑ may be I, or at least one of R₁₁ₐ to R₁₁ₑ, R₁₂ₐ, and R_{13b} may be at least one I;
b12a and b13a may each be an integer from 1 to 4;
A₁₁ and A₁₂ may each not exist, or may each be a benzene ring;
[symbol] may each be a carbon-carbon single bond or a carbon-carbon double bond;
L₁₁ may be a single bond, O, S, CO, SO, SO₂, CRR′, or NR;
R and R′ may each independently be hydrogen; deuterium; a halogen; a cyano group; or a hydroxyl group; or a linear, branched, or cyclic C₁-C₂₀ monovalent hydrocarbon group optionally containing a heteroatom; and
an adjacent two of R₁₁ₐ to R₁₁ₑ, R₁₂ₐ to R₁₂ₑ, and R₁₃ₐ to R₁₃ₑ may optionally be bonded to each other to form a condensed ring.

In one or more embodiments, in Formulae 1A-11 and 1A-12, at least one of R₁₁ₐ to R₁₁ₑ may be I.

In one or more embodiments, in Formulae 1A-11 and 1A-12, R₁₁ₐ, R_{11b}, R_{11c}, R₁₂ₐ, or R₁₃ₐ may be I.

In one or more embodiments, in Formulae 1A-11 and 1A-12, R_{11b} and R_{11c} may each be I, R_{11b} and R_{11d} may each be I, R₁₂ₐ and R_{11c} may each be I, or R₁₂ₐ and R₁₃ₐ may each be I.

In an embodiment, in Formula 1, A₁₁_{⁺} may be selected from group I:

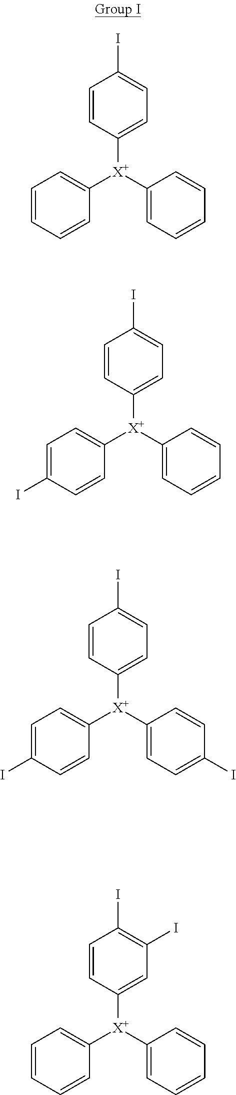

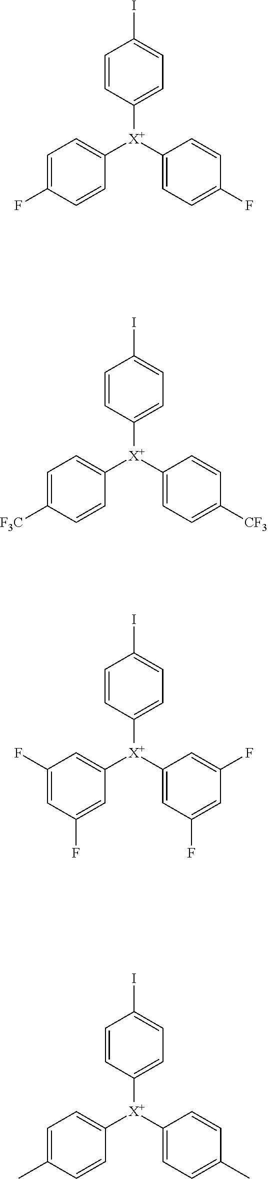

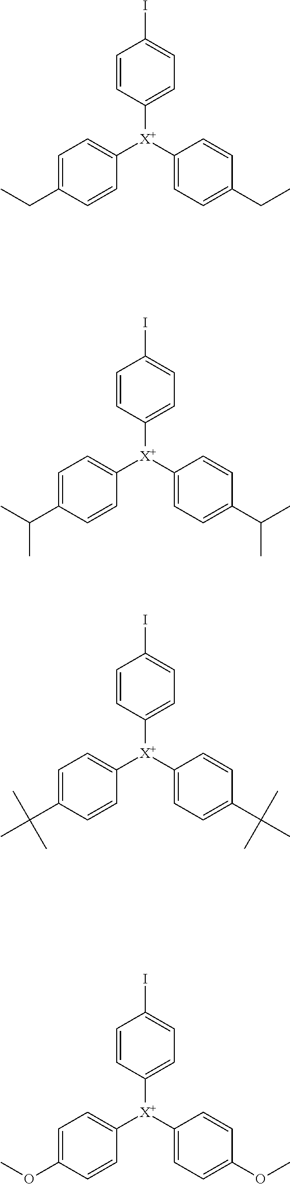

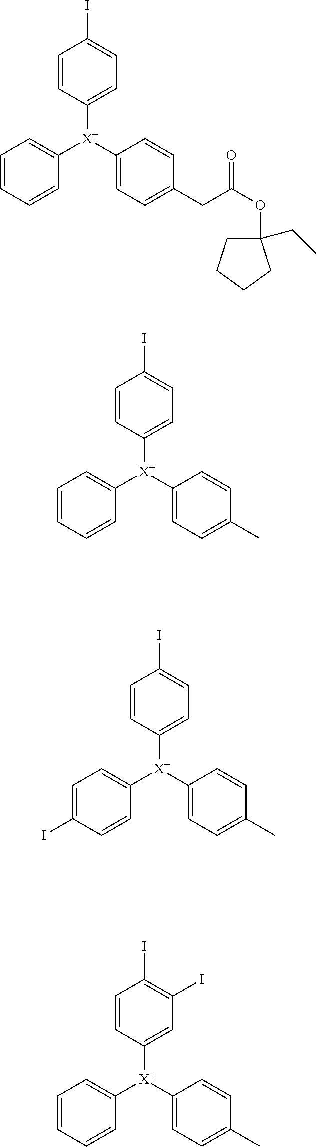

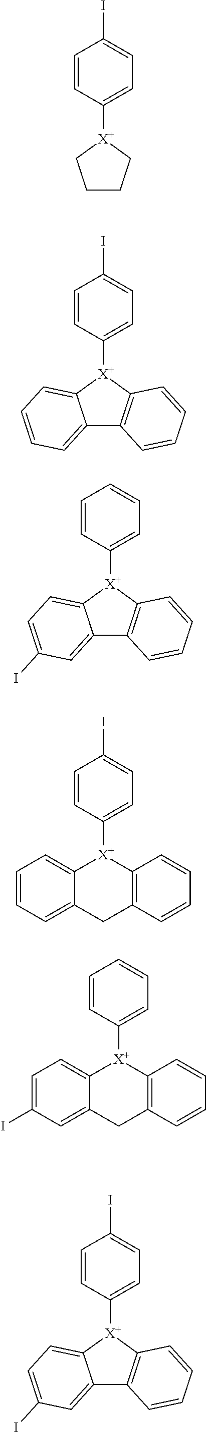

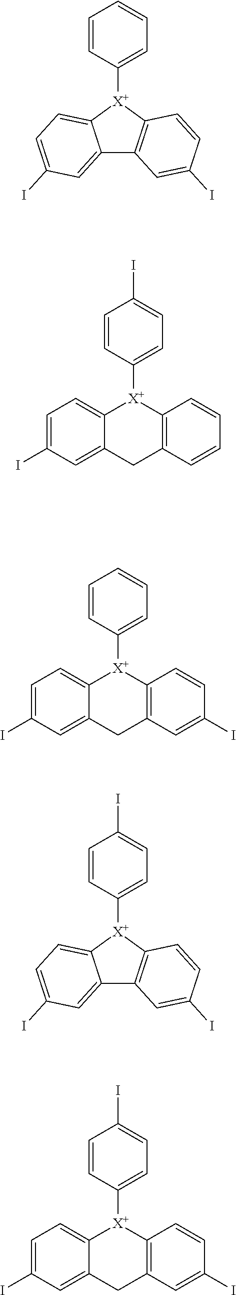

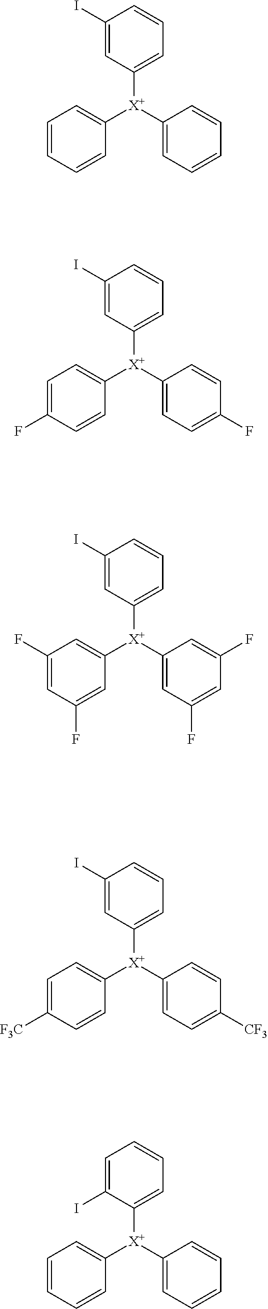

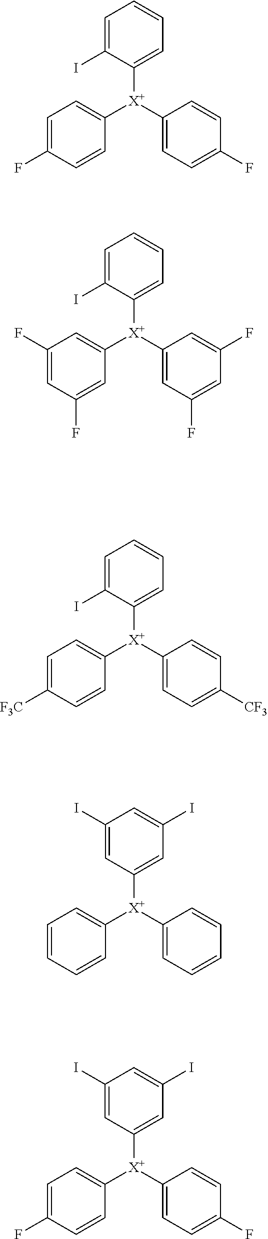

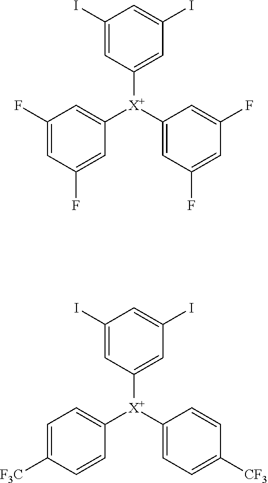

In Group I, X may be S, Se, or Te.

For example, in Formula 1B, L₂₁ and L₂₂ may each independently be a linear, branched, or cyclic C₁-C₃₀ monovalent hydrocarbon group optionally containing a single bond, O, C(═O), C(═O)O, OC(═O), C(═O)NH, NHC(═O), or a heteroatom.

In one or more embodiments, in Formula 1B, L₂₁ and L₂₂ may each independently be a single bond, 0, C(═O), C(═O)O, OC(═O), C(═O)NH, NHC(═O), a substituted or unsubstituted C₁-C₃₀ alkylene group, a substituted or unsubstituted C₃-C₂₀ cycloalkylene group, a substituted or unsubstituted C₃-C₂₀ heterocycloalkylene group, a substituted or unsubstituted C₂-C₂₀ alkenylene group, a substituted or unsubstituted C₃-C₂₀ cycloalkenylene group, a substituted or unsubstituted C₃-C₂₀ heterocycloalkenylene group, a substituted or unsubstituted C₆-C₂₀ arylene group, or a substituted or unsubstituted C₁-C₂₀ heteroarylene group.

In one or more embodiments, in Formula 1B, L₂₁ and L₂₂ may each independently be selected from: a single bond; C(═O); C(═O)O; OC(═O); and a C₁-C₂₀ alkylene group, a C₃-C₂₀ cycloalkylene group, a C₃-C₂₀ heterocycloalkylene group, a C₂-C₂₀ alkenylene group, a C₃-C₂₀ cycloalkenylene group, a C₃-C₂₀ heterocycloalkenylene group, a C₆-C₂₀ arylene group, and a C₁-C₂₀ heteroarylene group, each unsubstituted or substituted with deuterium, a halogen, a cyano group, a hydroxyl group, an amino group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In one or more embodiments, in Formula 1B,
L₂₁ and L₂₂ may each independently be a single bond, C(═O), C(═O)O, OC(═O), or CQ₁Q₂, and
Q₁ and Q₂ may each independently be hydrogen; deuterium; a halogen; a cyano group; a hydroxyl group; a C₁-C₂₀ alkyl group; or a C₁-C₂₀ halogenated alkyl group.

For example, in Formula 1B, a21 and a22 may each independently be 1.

For example, in Formula 1B, R₂₁ may be selected from a cyclopentyl group, a cyclohexyl group, a cycloheptyl group, a cyclooctyl group, a bicyclo[3.1.0]hexyl group, a bicyclo[3.2.0]heptyl group, a bicyclo[3.3.0]octyl group, a bicyclo[4.3.0]nonyl group, a bicyclo[4.4.0]decanyl group, a bicyclo[1.1.1]pentyl group, a bicyclo[2.1.1]hexyl group, a bicyclo[2.2.1]heptyl group, a bicyclo[2.2.2]octyl group, an adamantanyl group, a tricyclo[5.2.1.02,6]decanyl group, and a tetracyclo[6.2.1.13,6.02,7]dodecanyl group, each optionally containing deuterium, a halogen, a hydroxyl group, a carbonyl group, a carboxyl group, an ether bond, an ester bond, a carbonate, a lactone ring, a carboxylic anhydride moiety, a haloalkyl moiety, or any combination thereof.

In one or more embodiments, in Formula 1B, R21 may be selected from: groups represented by Formulae 9-1 to 9-37; one of the groups represented by Formulae 9-1 to 9-37, in which at least one hydrogen is substituted with deuterium; one of the groups represented by Formulae 9-1 to 9-37, in which at least one hydrogen is substituted with —F; one of the groups represented by Formulae 9-1 to 9-37, in which at least one hydrogen is substituted with —OH; one of the groups represented by Formulae 9-1 to 9-37, in which at least one carbon is substituted with oxygen; and one of the groups represented by Formulae 9-1 to 9-37, in which at least one carbon is substituted with a carbonyl group:

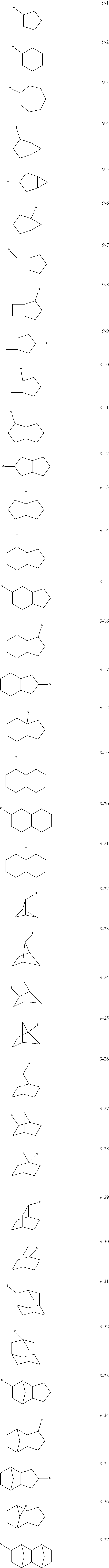

In Formulae 9-1 to 9-37,
* indicates a binding site to a neighboring atom.

In one or more embodiments, in Formula 1B, R21 may be selected from: the groups represented by Formulae 9-1 to 9-37; one of the groups represented by Formulae 9-1 to 9-37, in which at least one hydrogen is substituted with —F; one of the groups represented by Formulae 9-1 to 9-37, in which at least one hydrogen is substituted with —OH; and one of the groups represented by Formulae 9-1 to 9-37, in which at least one carbon is substituted with a carbonyl group.

For example, in Formula 1B, R22 may be selected from: hydrogen; deuterium; a halogen; and a C₁-C₂₀ alkyl group, a C₃-C₂₀ cycloalkyl group, and a C₆-C₂₀ aryl group, each unsubstituted or substituted with deuterium, a halogen, a cyano group, a hydroxyl group, a carboxyl group, an ester moiety, a sulfonate ester moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In one or more embodiments, in Formula 1B, R22 may be selected from: hydrogen; deuterium; a halogen; and a C₁-C₂₀ alkyl group, a C₃-C₂₀ cycloalkyl group, and a C₆-C₂₀ aryl group, each unsubstituted or substituted with deuterium, a halogen, a C₁-C₂₀ alkyl group, a C₁-C₃₀ halogenated alkyl group, a C₃-C₂₀ cycloalkyl group, a C₆-C₂₀ aryl group, or any combination thereof.

For example, in Formula 1B, R_{f} may be selected from F, CF₃, CF₂H, CFH₂, CH₂CF₃, CH₂CF₂H, CH₂CFH₂, CHFCH₃, CHFCF₂H, CHFCFH₂, CHFCF₃, CF₂CF₃, CF₂CF₂H, CF₂CFH₂, and one of groups represented by Formulae 10-1 to 10-14, in which at least one hydrogen is substituted with —F:

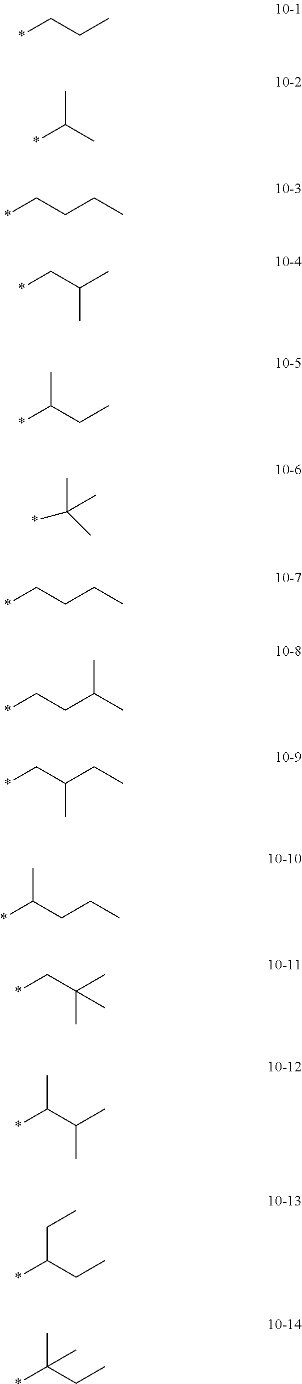

In Formulae 10-1 and 10-14, * indicates a binding site to a neighboring atom.

In one or more embodiments, in Formula 1B, R_{f} may be selected from F, CF₃, CF₂H, and CFH₂.

For example, in Formula 1B, c11 may be 4.

In an embodiment, in Formula 1, B₁₁⁻ may be represented by Formula 1B-1:

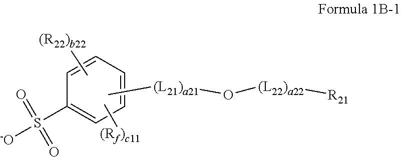

In Formula 1B-1, L₂₁, L₂₂, a21, a22, R₂₁, R₂₂, R_{f}, b22, and c11 may be referred to the descriptions thereof provided above.

In an embodiment, in Formula 1, B₁₁_{^{−}} may be represented by any one of Formulae 1B-11 and 1B-12:

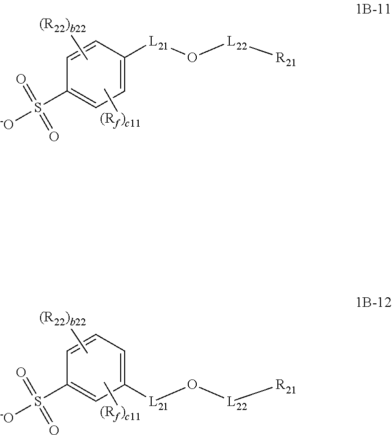

In Formulae 1B-11 and 1B-12, L₂₁, L₂₂, a21, a22, R₂₁, R₂₂, R_{f}, b22, and c11 may be referred to the descriptions thereof provided above.

In an embodiment, in Formula 1, B₁₁_{^{−}} may be selected from group II:

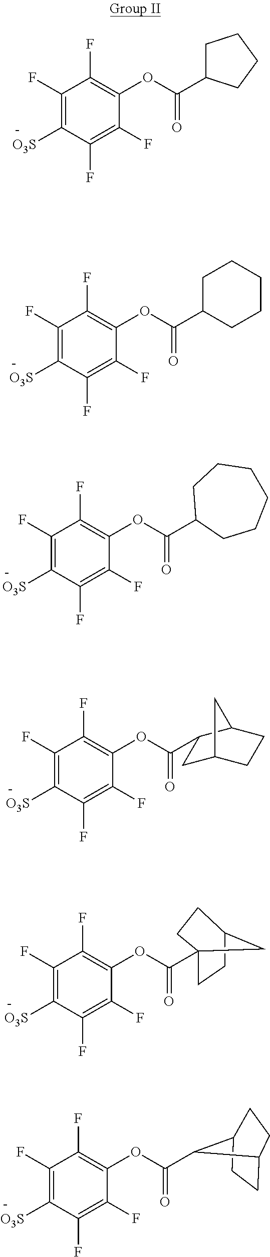

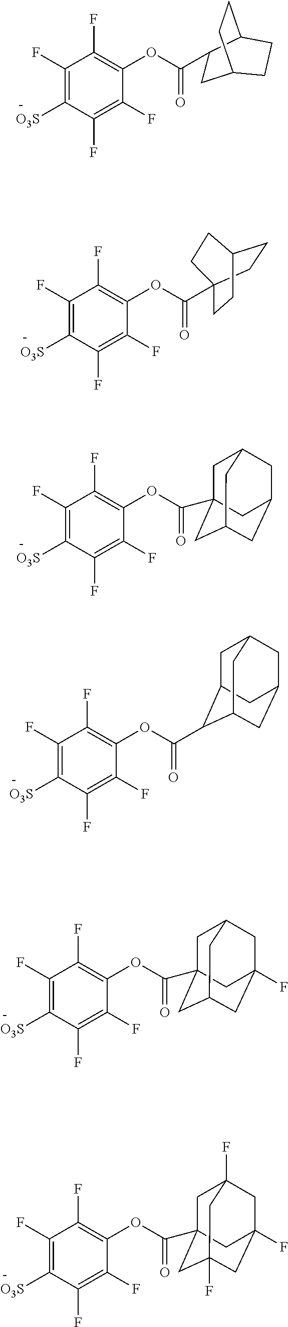

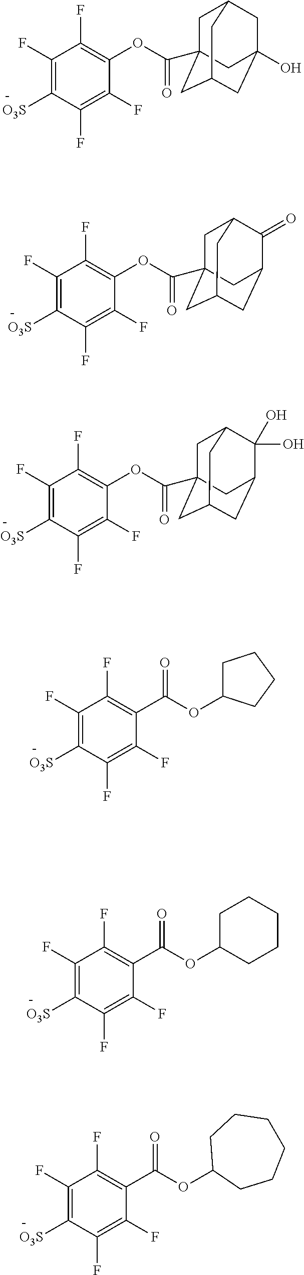

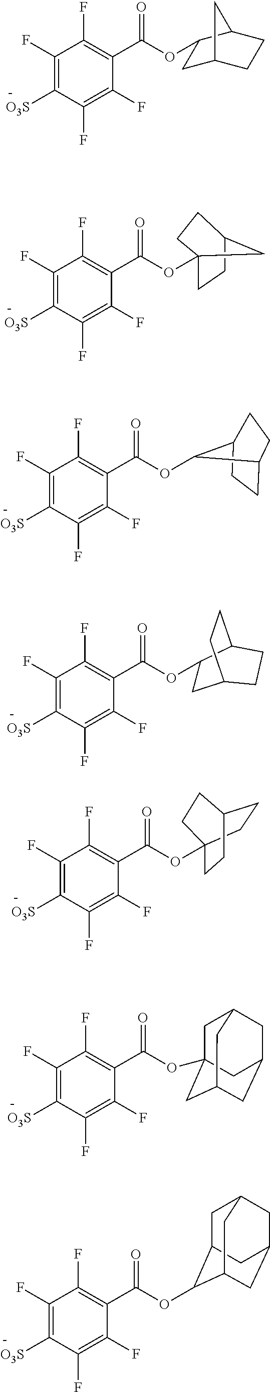

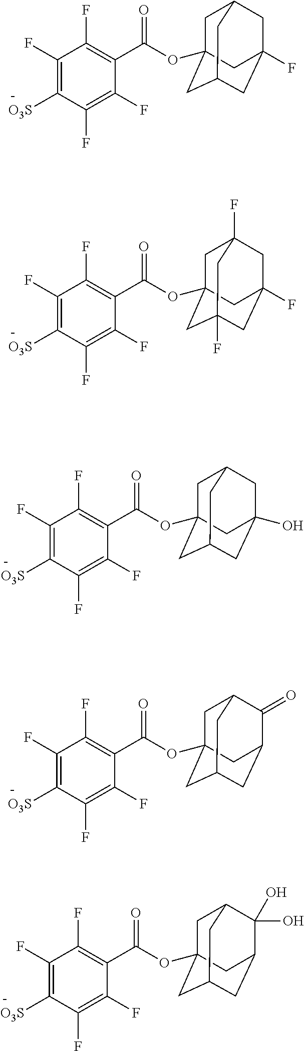

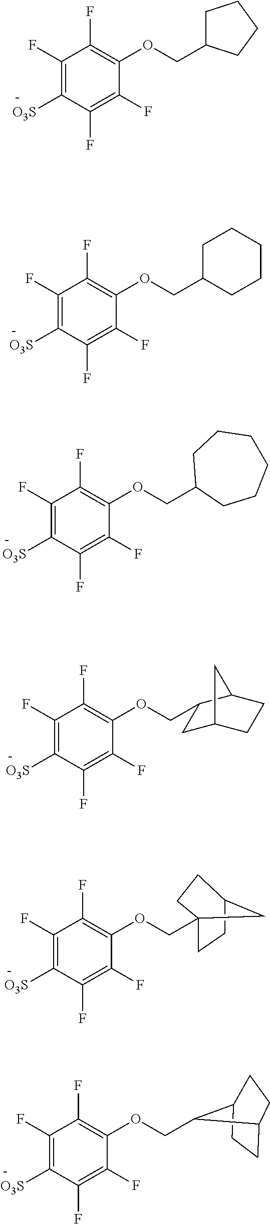

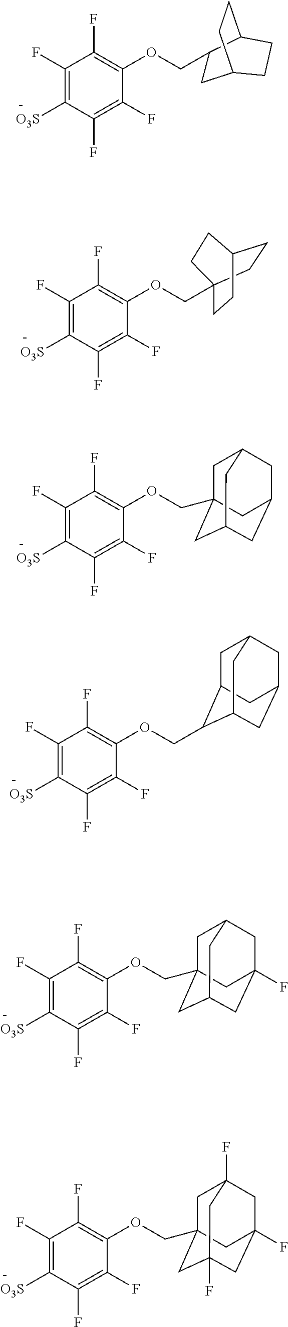

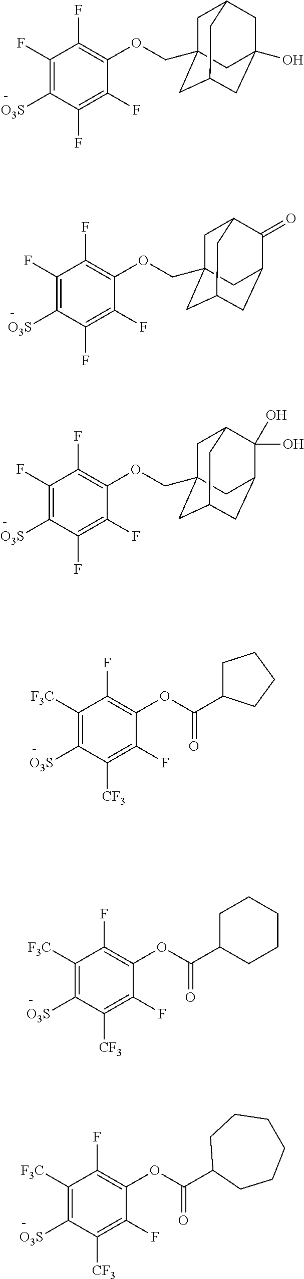

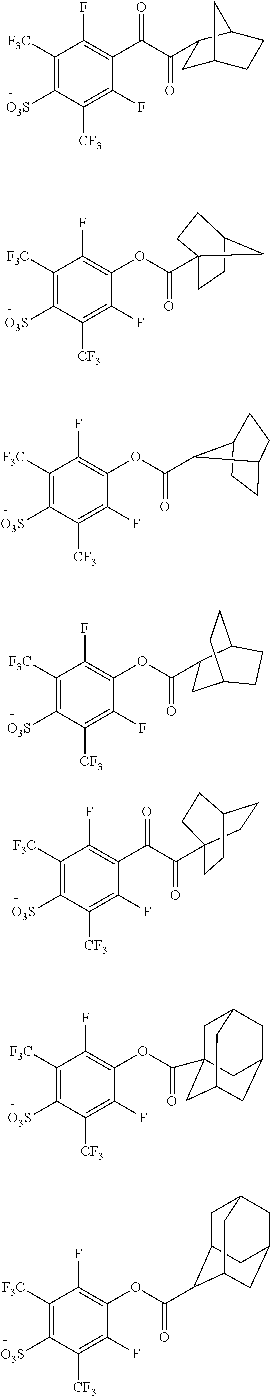

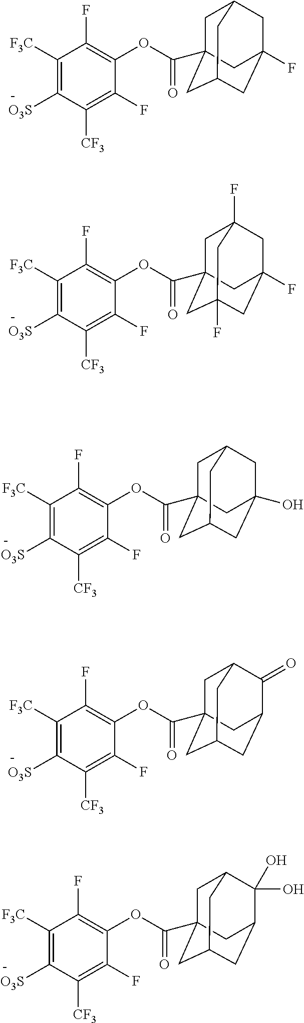

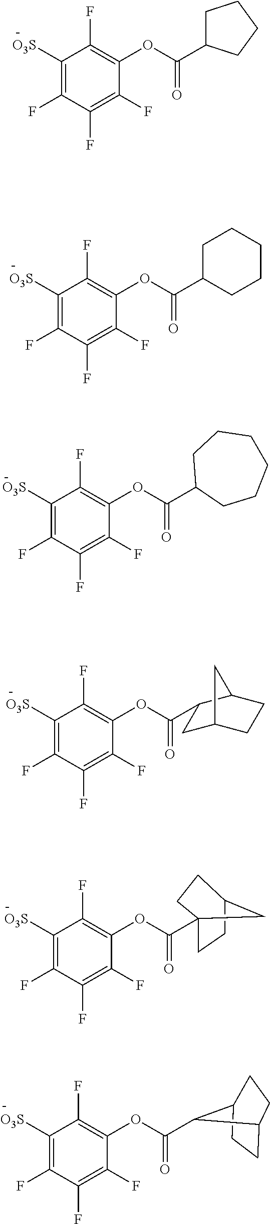

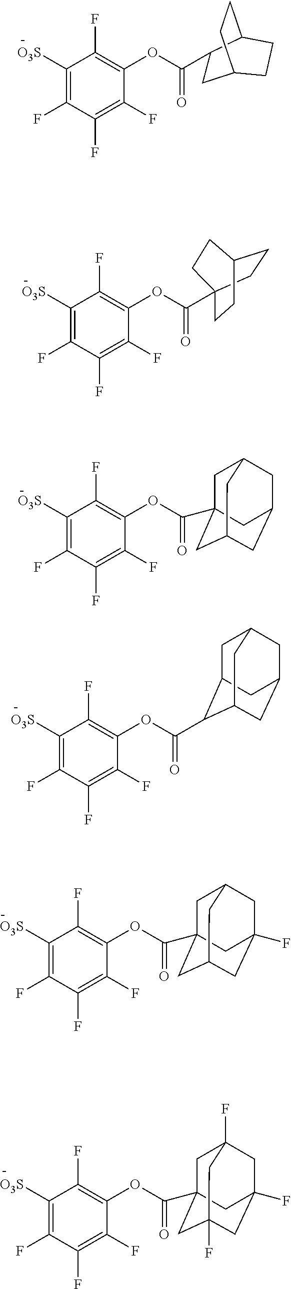

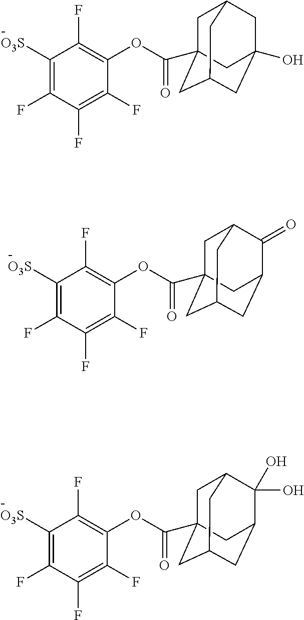

Generally, since an extreme ultraviolet (EUV) light source (13.5 nm) has a smaller number of photons compared to an ArF immersion light source, as the exposure dose decreases, noise may remarkably increase in the boundary area between an area exposed by the EUV light source and a non-exposed area. To compensate for this drawback, a lithography process by the EUV light source may require use of a greater amount of a photoacid generator compared to lithography processes by other light sources having the same amount of light. However, when the resist composition includes a large amount of the photoacid generator, the glass transition temperature (Tg) of a base resin may change, and thermal stability thereof may be deteriorated. In addition, the resolution of resist patterns formed by a photoacid generator remaining during the lithography process by the EUV light source may be reduced.

Compared to hydrogen, carbon, fluorine, chlorine, bromine, or the like, iodine has a remarkably high light absorption rate of about 1.4×10⁷ cm²/mol or more, and thus, the organic salt represented by Formula 1 necessarily containing at least one I may have enhanced absorbance.

In addition, since the acidity of benzenesulfonate is lower than that of alkylsulfonate, the organic salt represented by Formula 1 may have a relatively short effective acid diffusion length.

In addition, since the organic salt represented by Formula 1 necessarily contains at least one F, the organic salt may have a relatively high light absorption rate, for example, a relatively high EUV absorption rate, and may provide improved resolution due to low solubility (dark-loss) in an etchant.

Accordingly, even in a case in which a resist composition including the organic salt represented by Formula 1 has a relatively small amount of the organic salt represented by Formula 1, for example, the same or smaller amount of the organic salt of Formula 1 than when light sources other than EUV rays are used, when a pattern is formed through an EUV light source, a pattern with improved resolution may be provided.

The organic salt represented by Formula 1 maintains a low molecular weight since R₂₁ is not further substituted with a relatively large substituent such as an aryl group, and thus may exhibit effective properties even when only a relatively small amount of a photoacid generator is used.

In addition, the organic salt represented by Formula 1 may have a short effective acid diffusion length by limiting and/or minimizing molecular flexibility due to a long linear structure and a plurality of linkers.

### [Resist Composition]

According to one or more embodiments, a resist composition includes any one of the organic salts as described above, an organic solvent, and a base resin. The resist composition may have properties such as improved developability and/or improved resolution.

The resist composition undergoes a change in solubility in a developer by exposure to high-energy rays. The resist composition may be a positive resist composition in which an exposed portion of a resist film is dissolved and removed to form a positive resist pattern, or may be a negative resist composition in which a non-exposed portion of the resist film is dissolved and removed to form a negative resist pattern. In addition, a sensitive resist composition according to an embodiment may be for an alkaline developing process using an alkaline developer in the developing treatment to form a resist pattern, and may also be for a solvent developing process using a developer including an organic solvent (hereinafter, referred to as an organic developer) in the developing treatment.

The organic salt may be a photo-decomposable compound that can be decomposed by exposure to light. Since the organic salt can act as a photoacid generator when decomposed by exposure to light to generate an acid, the resist composition may not include an additional photoacid generator. Instead, the resist composition may further include a quencher. In one or more embodiments, the organic salt may be a photodegradable compound capable of generating an acid by exposure to light and serving as a quenching base to neutralize an acid before exposure to light. In this case, the organic salt may be used in combination with a photoacid generator that generates an acid. In addition, since the organic salt can generate an acid by exposure to light, the quencher function is lost by neutralization with the acid generated thereby, and thus the contrast between the exposed portion and the non-exposed portion may further be enhanced.

The organic salt may be used in an amount of about 0.1 parts by weight to about 40 parts by weight, for example, about 5 parts by weight to about 30 parts by weight, with respect to 100 parts by weight of the base resin. When the amount of the organic salt is within the above range, the function of the photoacid generator is exhibited at an appropriate level, and any performance loss, for example, the formation of foreign particles due to a decrease in sensitivity and/or lack of solubility may be reduced.

The organic salt is the same as described above, and thus, the organic solvent, the base resin, and optional components such as a photoacid generator to be contained as necessary will be described below. In addition, the organic salt represented by Formula 1 used in the resist composition may be used alone or a combination of at least two types of the organic salts represented by Formula 1 may also be used.

### <Organic Solvent>

The organic solvent included in the resist composition is not particularly limited as long as the organic solvent is capable of dissolving or dispersing an organic salt, a base resin, and optional components such as a photoacid generator to be included as necessary. The organic solvent may be used alone, or a combination of at least two types of organic solvents may also be used. In addition, a mixed solvent in which water and an organic solvent are mixed may be used.

Non-limiting examples of the organic solvent may include an alcohol-based solvent, an ether-based solvent, a ketone-based solvent, an amide-based solvent, an ester-based solvent, a sulfoxide-based solvent, and a hydrocarbon-based solvent.

Non-limiting examples of the alcohol-based solvent may include: monoalcohol-based solvents such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxybutanol, n-hexanol, 2-methylpentanol, sec-hexanol, 2-ethylbutanol, 4-methyl-2-pentanol (MIBC), sec-heptanol, 3-heptanol, n-octanol, 2-ethylhexanol, sec-octanol, n-nonyl alcohol, 2,6-dimethyl-4-heptanol, n-decanol, sec-undecyl alcohol, trimethylnonyl alcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, and diacetone alcohol; polyhydric alcohol-based solvents such as ethylene glycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethylene glycol, dipropylene glycol, triethylene glycol, tripropylene glycol, and the like; and polyhydric alcohol-containing ether-based solvents such as ethylene glycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, diethylene glycol dimethyl ether, propylene glycol monomethyl ether, propylene glycol dimethyl ether, propylene glycol monoethyl ether, propylene glycol monopropyl ether, propylene glycol monobutyl ether, dipropylene glycol monomethyl ether, dipropylene glycol monoethyl ether, dipropylene glycol monopropyl ether.

Non-limiting examples of the ether-based solvent may include: dialkyl ether-based solvents such as diethyl ether, dipropyl ether, and dibutyl ether; cyclic ether-based solvents such as tetrahydrofuran and tetrahydropyran; and aromatic ring-containing ether-based solvents such as diphenyl ether and anisole.

Non-limiting examples of the ketone-based solvent may include: chain ketone-based solvents such as acetone, methyl ethyl ketone, methyl-n-propyl ketone, methyl-n-butyl ketone, methyl-n-pentyl ketone, diethyl ketone, methyl isobutyl ketone, 2-heptanone, ethyl-n-butyl ketone, methyl-n-hexyl ketone, diisobutyl ketone, and trimethyl nonanone; cyclic ketone-based solvents such as cyclopentanone, cyclohexanone, cycloheptanone, cyclooctanone, and methylcyclohexanone; 2,4-pentanedione; acetonylacetone; and acetphenone.

Non-limiting examples of the amide-based solvent may include: cyclic amide-based solvents such as N,N′-dimethylimidazolidinone and N-methyl-2-pyrrolidone; and chain amide-based solvents such as N-methylformamide, N,N-dimethylformamide, N,N-diethylformamide, acetamide, N-methylacetamide, N,N-dimethylacetamide, and N-methylpropionamide.

Non-limiting examples of the ester-based solvent may include: acetate ester-based solvents such as methyl acetate, ethyl acetate, n-propyl acetate, isopropyl acetate, n-butyl acetate, isobutyl acetate, sec-butyl acetate, t-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, and n-nonyl acetate; polyhydric alcohol-containing ether carboxylate-based solvents such as ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate (PGMEA), propylene glycol monoethyl ether acetate, propylene glycol monopropyl ether acetate, propylene glycol monobutyl ether acetate, dipropylene glycol monomethyl ether acetate, and dipropylene glycol monoethyl ether acetate; lactone-based solvents such as γ-butyrolactone and 5-valerolactone; carbonate-based solvents such as dimethyl carbonate, diethyl carbonate, ethylene carbonate, and propylene carbonate; lactate ester-based solvents such as methyl lactate, ethyl lactate, n-butyl lactate, and n-amyl lactate; glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, and diethyl phthalate.

Non-limiting examples of the sulfoxide-based solvent may include dimethyl sulfoxide and diethyl sulfoxide.

Non-limiting examples of the hydrocarbon-based solvent may include: aliphatic hydrocarbon-based solvents such as n-pentane, isopentane, n-hexane, isohexane, n-heptane, isoheptane, 2,2,4-trimethylpentane, n-octane, isooctane, cyclohexane, and methylcyclohexane; and aromatic hydrocarbon-based solvents such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, and n-amylnaphthalene.

In one or more embodiments, the organic solvent may be selected from an alcohol-based solvent, an amide-based solvent, an ester-based solvent, a sulfoxide-based solvent, and any combination thereof. In one or more embodiments, the solvent may be selected from propylene glycol monomethyl ether, propylene glycol monoethyl ether, propylene glycol monomethyl ether acetate, N-methyl-2-pyrrolidone, N,N-dimethylacetamide, ethyl lactate, dimethyl sulfoxide, and any combination thereof.

In one or more embodiments, when an acid labile group in the form of acetal is used, a high-boiling alcohol such as diethylene glycol, propylene glycol, glycerol, 1,4-butanediol, or 1,3-butanediol may further be added to accelerate the deprotection reaction of acetal.

The organic solvent may be used in an amount of about 200 parts by weight to about 5,000 parts by weight, for example, about 400 parts by weight to about 3,000 parts by weight, with respect to 100 parts by weight of the base resin.

### <Base Resin>

The base resin may include a repeating unit represented by Formula 4 and containing an acid labile group:

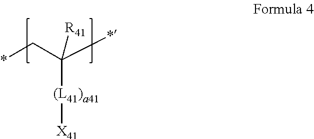

wherein, in Formula 4,
R₄₁ may be hydrogen, deuterium, a halogen, a linear or branched C₁-C₃₀ alkyl group, or a linear or branched C₁-C₃₀ halogenated alkyl group;
L₄₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, or a substituted or unsubstituted C₁-C₃₀ heteroarylene group;
a41 may be an integer selected from 1 to 6;
X₄₁ may be an acid labile group; and
* and *′ each indicate a binding site to a neighboring site.

For example, in Formula 4, R₄₁ may be hydrogen, deuterium, halogen, CH₃, CH₂F, CHF₂, or CF₃.

In Formula 4, L₄₁ may be selected from: a single bond; O; C(═O); C(═O)O; OC(═O); C(═O)NH; NHC(═O); and a C₁-C₃₀ alkylene group, a C₃-C₃₀ cycloalkylene group, a C₃-C₃₀ heterocycloalkylene group, a C₂-C₃₀ alkenylene group, a C₃-C₃₀ cycloalkenylene group, a C₃-C₃₀ heterocycloalkenylene group, a C₆-C₃₀ arylene group, and a C₁-C₃₀ heteroarylene group, each unsubstituted or substituted with deuterium, a halogen, a cyano group, a hydroxyl group, an amino group, a carboxylic acid group, an ester moiety, a sulfonate ester moiety, a carbonate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

In Formula 4, a41 refers to the number of repetitions of L₄₁, and when a41 is 2 or more, two or more of L₄₁ may be identical to or different from each other.

In an embodiment, in Formula 4, X₄₁ may be represented by any one of Formulae 6-1 to 6-7:

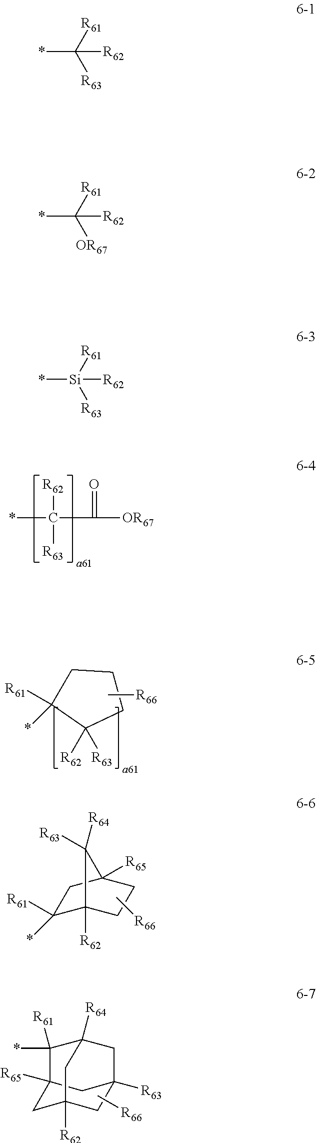

wherein, in Formulae 6-1 to 6-7,
A61 may be an integer from 0 to 6;
R₆₁ to R₆₆ may each independently be selected from hydrogen, deuterium, a halogen, a cyano group, a hydroxyl group, an amino group, a carboxylic acid group, and a linear, branched, or cyclic C₁-C₂₀ monovalent hydrocarbon group that may optionally contain a heteroatom;
R₆₇ may be a linear, branched, or cyclic C₁-C₂₀ monovalent hydrocarbon group optionally containing a heteroatom;
two adjacent groups of R₆₁ to R₆₇ may optionally be bonded to each other to form a ring; and
* indicates a binding site to a neighboring atom.

In Formulas 6-4 and 6-5, when a61 is 0, (CR₆₂R₆₃)ₐ₆₁ may be a single bond.

In an embodiment, the repeating unit represented by Formula 4 may be represented by any one of Formulae 4-1 and 4-2:

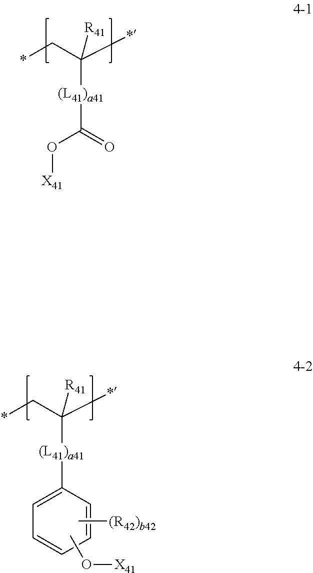

In Formulae 4-1 and 4-2,
L₄₁ and X₄₁ may be referred to the description thereof provided above;
a41 may be an integer from 1 to 4;
R₄₂ may be hydrogen, or a linear, branched, or cyclic monovalent hydrocarbon group optionally containing a heteroatom;
b42 may be integer from 1 to 4; and
* and *′ each indicate a binding site to a neighboring site.

The base resin including the repeating unit represented by Formula 4 decomposes under the action of an acid to generate a carboxyl group, thereby becoming alkali-soluble.

The base resin may further include, in addition to the repeating unit represented by Formula 4, a repeating unit represented by Formula 5:

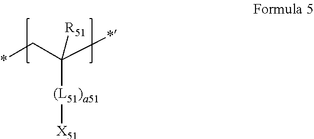

In Formula 5,
R₅₁ may be hydrogen, deuterium, a halogen, a linear or branched C₁-C₃₀ alkyl group, or a linear or branched C₁-C₃₀ halogenated alkyl group;
L₅₁ may be a single bond, O, C(═O), C(═O)O, OC(═O), C(═O)NH, NHC(═O), a substituted or unsubstituted C₁-C₃₀ alkylene group, a substituted or unsubstituted C₃-C₃₀ cycloalkylene group, a substituted or unsubstituted C₃-C₃₀ heterocycloalkylene group, a substituted or unsubstituted C₂-C₃₀ alkenylene group, a substituted or unsubstituted C₃-C₃₀ cycloalkenylene group, a substituted or unsubstituted C₃-C₃₀ heterocycloalkenylene group, a substituted or unsubstituted C₆-C₃₀ arylene group, or a substituted or unsubstituted C₁-C₃₀ heteroarylene group;
a51 may be an integer from 1 to 6;
X₅₁ may be a non-acid labile group; and
* and *′ each indicate a binding site to a neighboring site.

For example, R₅₁ in Formula 5 may be understood by referring to the description of R₄₁ in Formula 4.

L₅₁ in Formula 5 may be understood by referring to the description of L₄₁ in Formula 4.

In Formula 5, a51 refers to the number of repetitions of L₅₁, and when a51 is 2 or more, two or more of L₅₁ may be identical to or different from each other.

In an embodiment, X₅₁ in Formula 5 may be hydrogen, or a linear, branched, or cyclic C₁-C₂₀ monovalent hydrocarbon group containing at least one polar moiety selected from a hydroxyl group, a halogen, a cyano group, a carbonyl group, a carboxyl group, O, C(═O)O, OC(═O), S(═O)O, OS(═O), a lactone ring, a sultone ring, and a carboxylic anhydride moiety.

In an embodiment, the repeating unit represented by Formula 5 may be represented by any one of Formulae 5-1 and 5-2:

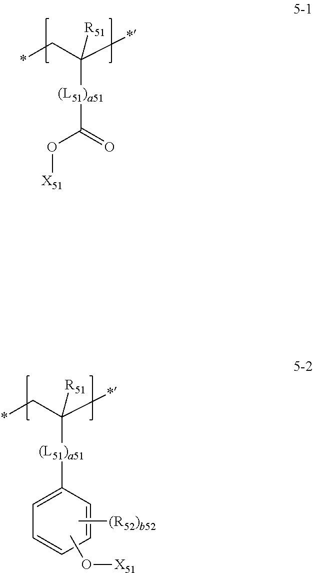

In Formulae 5-1 and 5-2,
L₅₁ and X₅₁ may be referred to the description thereof provided above;
a51 may be an integer from 1 to 4;
R₅₂ may be hydrogen or a hydroxyl group, or a linear, branched, or cyclic C₁-C₂₀ monovalent hydrocarbon group optionally containing a heteroatom;
b52 may be an integer from 1 to 4; and
* and *′ each indicate a binding site to a neighboring site.

For example, in an ArF lithography process, X₅₁ may contain a lactone ring as a polar moiety, and in KrF, EB and EUV lithography processes, X₅₁ may be a phenol.

In an embodiment, the base resin may further include a moiety containing an anion and/or a cation. For example, the base resin may further contain a moiety allowing a photoacid generator and/or a quencher to bind to the side chain thereof.

The base resin may have a weight average molecular weight (Mw) of about 1,000 to about 500,000, for example, about 3,000 to about 100,000, as measured by gel permeation chromatography using tetrahydrofuran as a solvent and polystyrene as a standard material.

The polydispersity index (PDI: Mw/Mn) of the base resin may be in a range of about 1.0 to about 3.0, for example, about 1.0 to about 2.0. When the PDI of the base resin is within the above range, the possibility of foreign matter remaining on a pattern may be reduced, or the deterioration of a pattern profile may be minimized. Accordingly, the resist composition may be more suitable for forming a micropattern.

The base resin may be prepared by any appropriate method, for example, by dissolving unsaturated bond-containing monomer(s) in an organic solvent, and then thermally polymerizing the resulting solution in the presence of a radical initiator.

In the base resin, the molar fraction (mol %) of each repeating unit derived from each monomer is as follows, but the disclosure is not limited thereto:
i) including about 1 mol % to about 60 mol %, for example, about 5 mol % to about 50 mol %, for example, about 10 mol % to about 50 mol %, of the repeating unit represented by Formula 4; and
ii) including about 40 mol % to about 99 mol %, for example, about 50 mol % to about 95 mol %, for example, about 50 mol % to about 90 mol %, of the repeating unit represented by Formula 5.

The base resin may be a homopolymer or may include a mixture of at least two polymers having different compositions, weight average molecular weights, and/or polydispersity indices.

### <Photoacid Generator>

Since the organic salt can act as a photoacid generator when decomposed by exposure to light to generate an acid, the resist composition may not include an additional photoacid generator.

### <Quencher>

The quencher may be a salt that generates an acid having a weaker acidity than the acid generated from the organic salt represented by Formula 1 and/or the photoacid generator.

The quencher may include an ammonium salt, a sulfonium salt, an iodonium salt, and a combination thereof.

In an embodiment, the quencher may be represented by Formula 8:

B₈₁_{⁺}A₈₁_{^{−}} Formula 8

In Formula 8,
B₈₁_{⁺} may be represented by any one of Formulae 8A to 8C, and A₈₁_{^{−}} may be represented by any one of Formulae 8D to 8F;
B₈₁_{⁺} and A₈₁_{^{−}} may optionally be linked to each other via a carbon-carbon covalent bond;

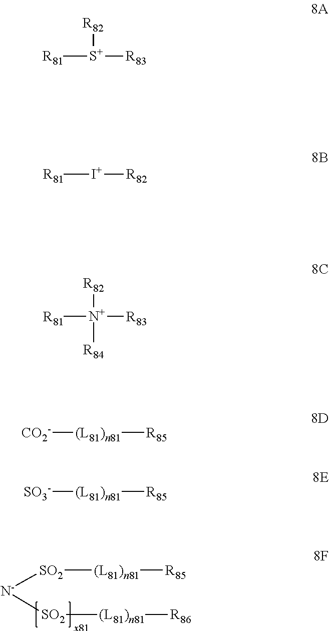

In Formulae 8A to 8F,
L₈₁ and L₈₂ may each independently be a single bond or CRR′;
R and R′ may each independently be hydrogen, deuterium, a halogen, a cyano group, a hydroxyl group, a C₁-C₂₀ alkyl group, a C₁-C₂₀ halogenated alkyl group, a C₁-C₂₀ alkoxy group, a C₃-C₂₀ cycloalkyl group, or a C₃-C₂₀ cycloalkoxy group;
n81 and n82 may each independently be 1, 2, or 3;
x81 may be 0 or 1;
R₈₁ to R₈₄ may each independently be a linear, branched, or cyclic C₁-C₂₀ monovalent hydrocarbon group;
two adjacent groups of R₈₁ to R₈₄ may optionally be bonded to each other to form a ring; and
R₈₅ and R₈₆ may be: hydrogen; a halogen; or a linear, branched, or cyclic C₁-C₂₀ monovalent hydrocarbon group optionally containing a heteroatom.

The quencher may be included in an amount of about 0.01 parts by weight to about 10 parts by weight, about 0.05 parts by weight to about 5 parts by weight, or about 0.1 parts by weight to about 3 parts by weight, with respect to 100 parts by weight of the base resin. When the amount of the quencher is within the above range, appropriate resolution may be achieved, and complications related to foreign particles after developing or during stripping may be reduced.

The quencher may be used alone, or a combination of at least two types of quenchers may also be used.

### <Optional Components>

The resist composition may further include a surfactant, a crosslinking agent, a leveling agent, a colorant, or any combination thereof as necessary.

The resist composition may further include a surfactant to improve applicability, developability, and the like. Non-limiting examples of the surfactant may include nonionic surfactants such as polyoxyethylene lauryl ether, polyoxyethylene stearyl ether, polyoxyethylene oleyl ether, polyoxyethylene n-octylphenyl ether, polyoxyethylene n-nonylphenyl ether, polyethylene glycol dilaurate, and polyethylene glycol distearate. As the surfactant, a commercially available product or a synthetic product may be used. Non-limiting examples of the commercially available surfactant product may include KP341 (manufactured by Shin-Etsu Chemical Co., Ltd.), Polyflow No. 75, Polyflow No. 95 (manufactured by Kyoeisha Chemical Co., Ltd.), FTOP EF301, FTOP EF303, FTOP EF352 (manufactured by Mitsubishi Material Electron Chemical Co., Ltd.), MEGAFACE (registered trademark) F171, MEGAFACE F173, R40, R41, R43 (manufactured by DIC Corporation), FLUORAD (registered trademark) FC430, Fluorad FC431 (manufactured by 3M), AsahiGuard AG710 (manufactured by AGC Corporation), SURFLON (registered trademark) S-382, SURFLON SC-101, SURFLON SC-102, SURFLON SC-103, SURFLON SC-104, SURFLON SC-105, and SURFLON SC-106 (manufactured by AGC Seimi Chemical Co., Ltd.).

The surfactant may be included in an amount of about 0 parts by weight to about 20 parts by weight with respect to 100 parts by weight of the base resin. The surfactant may be used alone, or a combination of at least two types of surfactants may also be used.

A method of preparing the resist composition is not particularly limited, and the resist composition may be prepared by, for example, mixing, in an organic solvent, an organic salt, a base resin, a photoacid generator, and optional components added as needed. The temperature or time during mixing is not particularly limited. If necessary, filtration may be performed after mixing.

### [Pattern Forming Method]

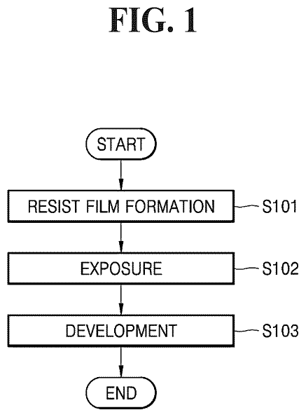

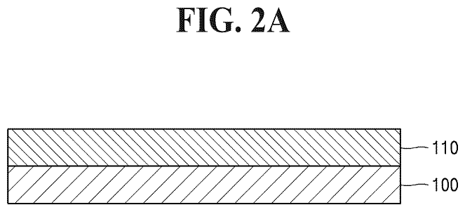

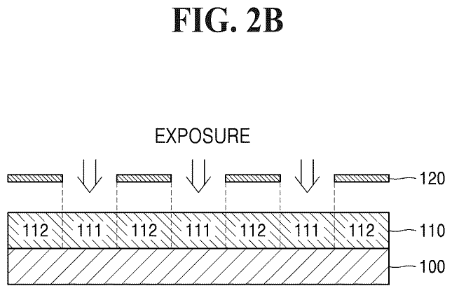

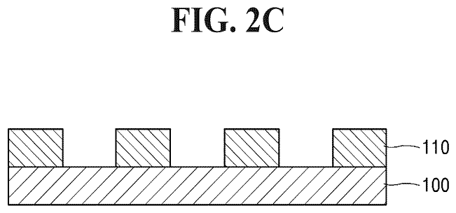

Hereinafter, a method of forming a pattern according to embodiments will be described in more detail with reference to FIGS. 1 and 2A to 2C. FIG. 1 is a flowchart illustrating a method of forming a pattern according to embodiments. FIG. 2A to 2C are side cross-sectional views illustrating a method of forming a pattern according to embodiments. Hereinafter, a method of forming a pattern using a positive resist composition will be described in detail as an example, but the disclosure is not limited thereto.

Referring to FIG. 1, the method of forming a pattern includes a process of forming a resist film by applying a resist composition (S101), a process of exposing at least a portion of the resist film to high-energy rays (S102), and a process of developing the exposed resist film by using a developer (S103). The above processes may be omitted as necessary, or may be performed in an order different from the stated order.

First, a substrate 100 is prepared. As the substrate 100, for example, a semiconductor substrate such as a silicon substrate or a germanium substrate, glass, quartz, ceramic, copper, or the like may be used. In some embodiments, the substrate 100 may include a group Ill-V compound such as GaP, GaAs, GaSb, or the like.

A resist film 110 may be formed by applying the resist composition to a desired thickness on the substrate 100 through, for example, coating. If necessary, heating may be performed to remove the organic solvent remaining in the resist film 110. As the coating method, spin coating, dipping, roller coating, or other general coating methods may be used. Among these coating methods, spin coating may be used, and the resist film 110 having a certain thickness may be formed by adjusting the viscosity, concentration, and/or spin speed of the resist composition. In one or more embodiments, the resist film 110 may have a thickness of about 10 nm to about 300 nm. In one or more embodiments, the thickness of the resist film 110 may be in a range of about 30 nm to about 200 nm.

The lower limit of the prebaking temperature may be 60° C. or higher, for example, 80° C. or higher. In addition, the upper limit of the prebaking temperature may be 150° C. or less, for example, 140° C. or less. The lower limit of the prebaking time may be 5 seconds or longer, for example, 10 seconds or longer. The upper limit of the prebaking time may be 600 seconds or less, for example, 300 seconds or less.

Before the resist composition is applied on the substrate 100, a film to be etched (not shown) may further be formed on the substrate 100. The film to be etched may refer to a layer onto which an image is transferred from a resist pattern and which is converted into a certain pattern. In an embodiment, the film to be etched may be formed to include, for example, an insulating material such as silicon oxide, silicon nitride, or silicon oxynitride. In some embodiments, the film to be etched may be formed to include a conductive material such as a metal, a metal nitride, a metal silicide, or a metal silicide nitride film. In some embodiments, the film to be etched may be formed to include a semiconductor material such as polysilicon.

In an embodiment, an anti-reflective film may further be formed on the substrate 100 to maximize the efficiency of a resist. The anti-reflective film may be an organic or inorganic anti-reflective film.

In an embodiment, a protective film may further be provided on the resist film 100 to reduce the influence of alkaline impurities and the like included in the process.

In addition, in the case of immersion exposure, to avoid direct contact between an immersion medium and the resist film 100, for example, a protective film for immersion may be provided on the resist film 100.

Next, at least a portion of the resist film 110 may be exposed to high-energy rays. For example, high energy rays passing through a mask 120 may be irradiated to at least a portion of the resist film 110. Thus, the resist film 110 may have an exposed portion 111 and a non-exposed portion 112.

In some cases, the exposure to high-energy rays is performed by irradiating high-energy rays through a mask having a certain pattern using a liquid such as water as a medium. Non-limiting examples of the high-energy rays may include: electromagnetic waves such as ultraviolet rays, deep ultraviolet rays, extreme ultraviolet rays (EUV, wavelength 13.5 nm), X-rays, and γ-rays; and charged particle beams, such as an electron beam (EB) and an α-ray. Irradiation of these high-energy rays can be collectively referred to as “exposure to light.”

Exposure to light may be performed through light irradiation using various light sources, e.g., lasers in the ultraviolet region, such as a KrF excimer laser (wavelength: 248 nm), an ArF excimer laser (wavelength: 193 nm), and an F₂ excimer laser (wavelength: 157 nm), a harmonic laser that emits laser beams in the far ultraviolet region or vacuum ultraviolet region by converting the wavelength of laser beams from a solid-state laser light source (YAG, a semiconductor laser, or the like), and electron beams or extreme ultraviolet (EUV) rays. Generally, exposure to light may be performed through a mask corresponding to a desired pattern. However, when electron beams are used as a light source for exposure to light, exposure to light may be performed by direct drawing without using a mask.

When EUV rays are used as the high-energy rays, the high-energy rays may have an integral dose of, for example, 2,000 mJ/cm² or less, for example, 500 mJ/cm² or less. In addition, when electron beams are used as the high-energy rays, the integral dose of the high-energy rays may be 5,000 μC/cm² or less, for example, 1,000 μC/cm² or less.

In addition, post exposure bake (PEB) may be performed after exposure. The lower limit of the PEB temperature may be 50° C. or higher, for example, 80° C. or higher. The lower limit of the PEB temperature may be 180° C. or less, for example, 130° C. or less. The lower limit of the PEB time may be 5 seconds or more, for example, 10 seconds or more. The upper limit of the PEB time may be 600 seconds or less, for example, 300 seconds or less.

Next, the exposed resist film 110 may be developed by using a developer. The exposed portion 111 may be washed away by the developer, and the non-exposed portion 112 remains without being washed away by the developer.

Examples of the developer may include an alkaline developer and a developer including an organic solvent (hereinafter, also referred to as an “organic developer”). Non-limiting examples of the developing method may include a dipping method, a puddle method, a spray method, and a dynamic dosing method. The developing temperature may be, for example, in a range of about 5° C. to about 60° C., and the developing time may be, for example, in a range of about 5 seconds to about 300 seconds.

Non-limiting examples of the alkaline developer may include an aqueous alkaline solution obtained by dissolving at least one alkaline compound selected from sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethyl amine, ethyldimethylamine, triethanolamine, tetramethyl ammonium hydroxide (TMAH), pyrrole, piperidine, choline, 1,8-diazabicyclo[5.4.0]-7-undecene (DBU), and 1,5-diazabicyclo[4.3.0]-5-nonene (DBN). The alkaline developer may further include a surfactant.

The lower limit of the amount of an alkaline compound in the alkaline developer may be 0.1 mass % or more, for example, 0.5 mass % or more, for example, 1 mass % or more. In addition, the upper limit of the amount of the alkaline compound in the alkaline developer may be 20 mass % or less, for example, 10 mass % or less, for example, 5 mass % or less.

After developing, the resist pattern may be washed with ultrapure water, and then water remaining on the substrate and the pattern may be removed.

As an organic solvent included in the organic developer, the organic solvent as described above in <Organic solvent> of [Resist composition] may be used.

The lower limit of the amount of the organic solvent content in the organic developer may be 80 mass % or more, for example, 90 mass % or more, for example, 95 mass % or more, for example, 99 mass % or more.

The organic developer may include a surfactant. In addition, a trace amount of water may be included in the organic developer. In addition, in developing, the developing may be stopped by replacement with different types of solvents from the organic developer.

The resist pattern after developing may further be cleaned. Ultrapure water, rinse liquid, or the like may be used as a cleaning liquid. The rinse liquid is not particularly limited as long as it does not dissolve the resist pattern, and a solution including a general organic solvent may be used. For example, the rinse liquid may be an alcohol-based solvent or an ester-based solvent. After cleaning, the rinse liquid remaining on the substrate and the pattern may be removed. In addition, when ultrapure water is used, water remaining on the substrate and the pattern may be removed.

In addition, developers may be used alone, or a combination of at least two thereof may be used.

After the resist pattern is formed as described above, a patterned wiring board may be obtained by etching. Etching may be performed by known methods such as dry etching using a plasma gas and wet etching using an alkaline solution, a cupric chloride solution, a ferric chloride solution, or the like.

After the resist pattern is formed, plating may be performed. The plating method is not particularly limited, and examples thereof include copper plating, solder plating, nickel plating, gold plating, and the like.

The resist pattern remaining after etching may be peeled off using an organic solvent. Examples of such an organic solvent may include, but are not particularly limited to, propylene glycol monomethyl ether acetate (PGMEA), propylene glycol monomethyl ether (PGME), ethyl lactate (EL), and the like. The peeling method is not particularly limited, and may be, for example, a dipping method, a spray method, or the like. In addition, the wiring board on which the resist pattern is formed may be a multilayer wiring board or may have small-diameter through-holes.

In an embodiment, the wiring board may be formed by a method of depositing a metal in a vacuum after the resist pattern is formed, and then melting the resist pattern into a solution, that is, a lift-off method.

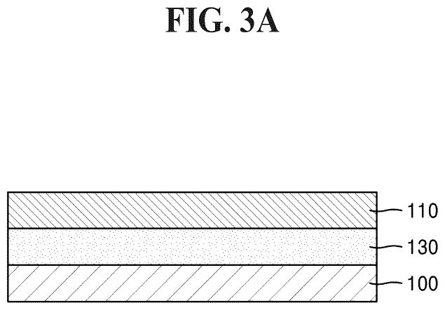

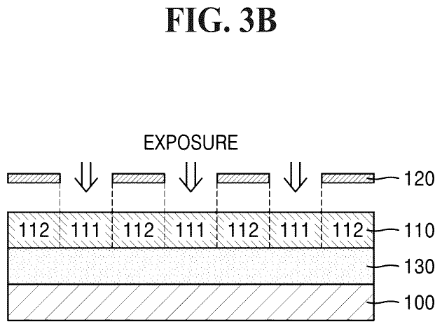

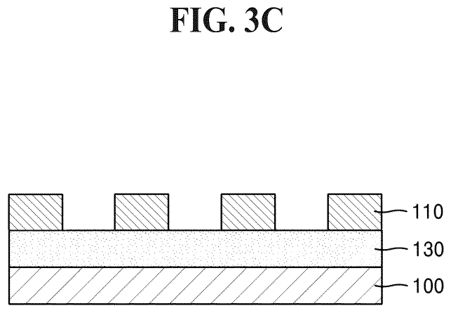

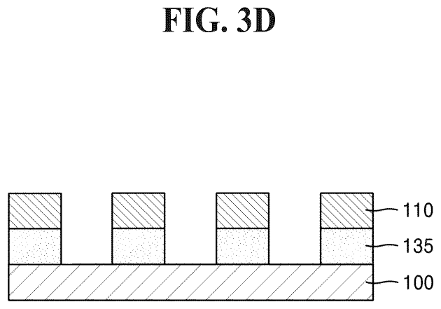

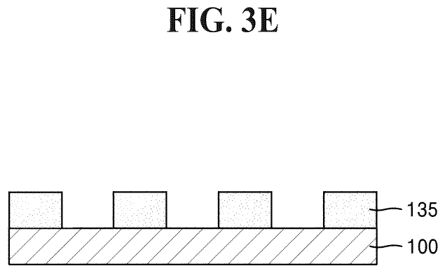

FIGS. 3A to 3E are side cross-sectional views illustrating a method of forming a patterned structure according to an embodiment.

Referring to FIG. 3A, a material layer 130 may be formed on the substrate 100 before forming a resist film 110 on the substrate 100. The resist film 110 may be formed on top of the material layer 130. The material layer 130 may include an insulating material (e.g., silicon oxide, silicon nitride), a semiconductor material (e.g., silicon), a metal (e.g., copper). In some embodiments, the material layer 130 may be a multi-layer structure. A material of the material layer 130 may be different than a material of the substrate 100.

Referring to FIG. 3B, the resist film 110 may undergo a pre-exposure bake process and may be exposed with high energy rays through a mask 120, after which the resist film 110 may include exposed regions 111 and unexposed regions 112.

Referring to FIG. 3C, the exposed resist film 110 may be developed using a developer (e.g., developing solution). The exposed area 111 may be washed away by the developer, whereas the unexposed area 112 may remain without being washed away by the developer.

Referring to FIG. 3D, exposed areas of the material layer 130 may be etched using the resist pattern 110 as a mask to form a material pattern 135 on the substrate 100.

Referring to FIG. 3E, the resist pattern 110 may be removed.

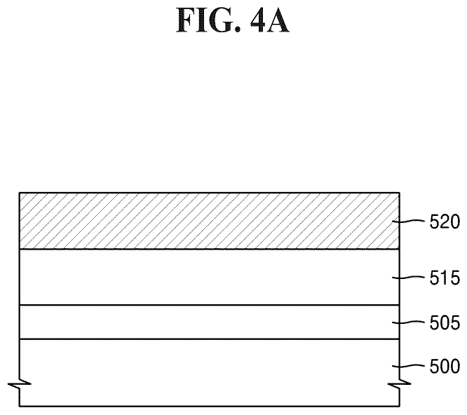

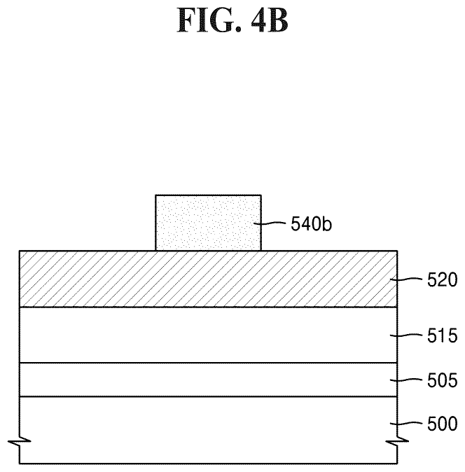

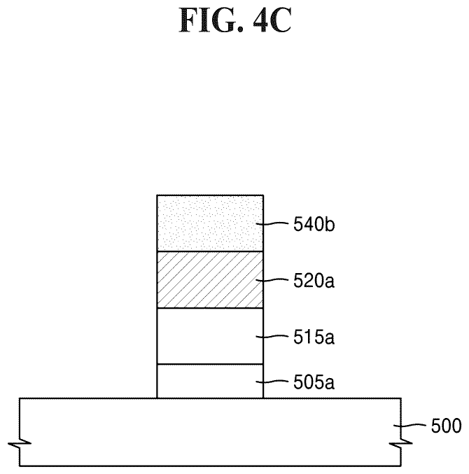

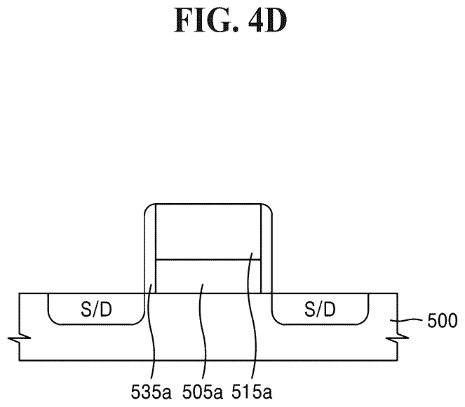

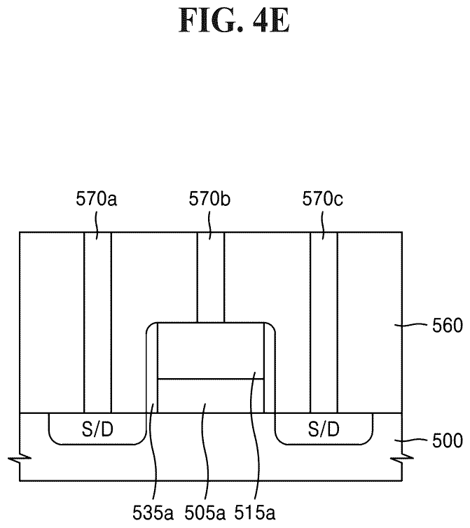

FIGS. 4A to 4E are side cross-sectional views illustrating a method of forming a semiconductor device according to an embodiment.

Referring to FIG. 4A, a gate dielectric 505 (e.g., silicon oxide) may be formed on a substrate 500. The substrate 500 may be a semiconductor substrate, such as a silicon substrate. A gate layer 515 (e.g., doped polysilicon) may be formed on the gate dielectric 505. A hardmask layer 520 may be formed on the gate layer 515.

Referring to FIG. 4B, a resist pattern 540b may be formed on the hardmask layer 520. The resist pattern 540b may be formed using a resist composition according to example embodiments. The resist composition may include a photoacid generator and an organic solvent.

Referring to FIG. 4C, the gate layer 515 and the gate dielectric 505 may be etched to form a hardmask pattern 520a, a gate electrode pattern 515a, and a gate dielectric pattern 505a.

Referring to FIG. 4D, a spacer layer may be formed over the gate electrode pattern 515a and the gate dielectric pattern 505a. The spacer layer may be formed using a deposition process (e.g., CVD). The spacer layer may be etched to form spacers 535a (e.g., silicon nitride) on sidewalls of the gate electrode pattern 515a and the gate dielectric pattern 505a. After forming the spacers 535a, ions may be implanted into the substrate 500 to form source/drain impurity regions S/D.

Referring to FIG. 4E, an interlayer insulating layer 560 (e.g., oxide) may be formed on the substrate 500 to cover the gate electrode pattern 515a, gate dielectric pattern 505a, and spacers 535a. Then, electrical contacts 570a, 570b, and 570c may be formed in the interlayer insulating layer 560 to connect to the gate electrode 515a and the S/D regions. The electrical contacts may be formed of a conductive material (e.g., metal). Although not illustrated, a barrier layer may be formed between sidewalls of the interlayer insulating layer 560 and the electrical contacts 570a, 570b, and 570c. While FIGS. 4A to 4E illustrate an example of forming a transistor, inventive concepts are not limited thereto. A resist composition including an organic salt according to one or more embodiments may be used in a patterning process to form other types of semiconductor devices. The resist composition may include a photoacid generator and an organic solvent.

The disclosure will be described in further detail with reference to the following examples and comparative examples. However, these examples are not intended to limit the technical scope of inventive concepts in the present disclosure.

### EXAMPLES

### Synthesis Example 1: Synthesis of Compound PAG A

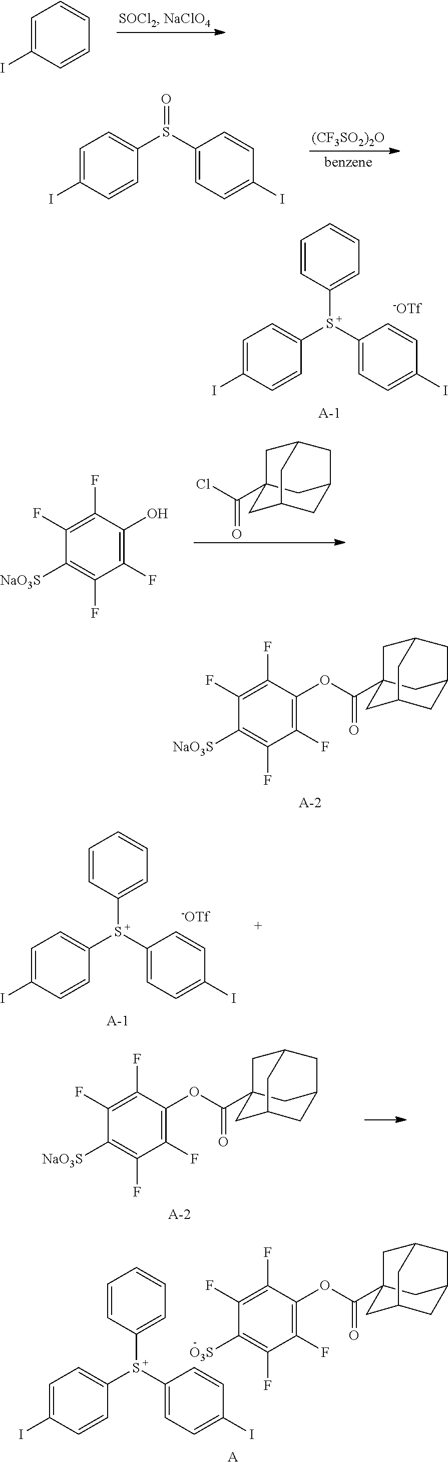

### Synthesis of Compound A-1

### (1) Synthesis of 4,4′-sulfinyl bisiodobenzene

4-iodobenzene (2.246 g, 11.01 mmol), thionyl chloride (0.655 g, 5.51 mmol), and sodium perchlorate (0.117 g, 1.10 mmol) were added into 12 ml of tetrahydrofuran, followed by stirring for 3 hours. Subsequently, the reaction solvent was removed through distillation under reduced pressure, and then an organic layer obtained through extraction with 30 ml of water and 30 ml of dichloromethane (DCM) was dried with Na₂SO₄ and filtered. The filtrate obtained therefrom was decompressed to obtain a residue, and the residue was separated and purified by column chromatography to thereby obtain 4,4′-sulfinylbis(iodobenzene). The produced compound was identified by nuclear magnetic resonance (NMR) and liquid chromatography-mass spectrometry (LC-MS).

¹H-NMR (500 MHz, CDCl₃): δ 7.05 (d, 4H), 7.42 (d, 4H), LC-MS m/z=454.85 (M+H)

### (2) Synthesis of Compound A-1

4,4′-sulfinylbis(iodobenzene) (3.73 g, 8.20 mmol) was dissolved in 15 ml of benzene, and then trifluoromethanesulfonic anhydride (2.778 g, 9.85 mmol) was added dropwise thereto at 0° C., followed by stirring at room temperature for 1 hour. Subsequently, an organic layer obtained through extraction with 20 ml of water and 50 ml of ethyl acetate was washed with an aqueous saturated NaHCO₃ solution, dried with MgSO₄, and filtered. The filtrate obtained therefrom was decompressed to obtain a residue, and the residue was separated and purified by silica gel column chromatography to thereby obtain compound A-1 (4.92 g, 90%). The produced compound was identified by NMR and LC-MS.

¹H-NMR (500 MHz, CD₂Cl₂): δ 8.08 (d, 4H), 7.84 (t, 1H), 7.74 (t, 2H), 7.69 (d, 2H), 7.41 (d, 2H), LC-MS m/z=514.88 (Cation)

### Synthesis of Compound A-2

1-adamantanecarbonyl chloride (1 g, 5.03 mmol) was dissolved in 50 ml of acetonitrile, and then 4-dimethylaminopyridine (0.64 g, 5.26 mmol) was added thereto, followed by stirring in an ice bath for 10 minutes. Then, sodium 2,3,5,6-tetrafluoro-4-hydroxybenzenesulfonate (1.41 g, 5.26 mmol) and triethylamine (0.74 ml, 5.26 mmol) were slowly added dropwise, and then a reaction was allowed to occur therebetween at room temperature for 2 hours. After the reaction was completed, the precipitate was removed by filtering, and then washed twice with brine in a separatory funnel, and the organic layer was column purified under a condition of DCM:MeOH=20:1 (volume ratio) to thereby obtain compound A-2 (1.8 g, 84%). The produced compound was identified by NMR and LC-MS.

¹H-NMR (500 MHz, CD₂Cl₂): δ 2.00 (m, 3H), 1.89 (m, 6H), 1.71 (m, 6H), LC-MS m/z=407.06 (anion).

### Synthesis of Compound PAG A

Compound A-1 (0.66 g, 1 mmol) and Compound A-3 (0.43 g, 1 mmol) were mixed with 10 ml of dichloromethane and 1 ml of water, followed by stirring for 4 hours. Subsequently, the organic layer was separated, dried with MgSO₄, and filtered, and then the filtrate obtained therefrom was decompressed to obtain a residue, and the residue was separated and purified by silica gel column chromatography to thereby obtain Compound PAG A (0.47 g, yield: 44.5%). The produced compound was identified by NMR and matrix assisted laser desorption/ionization (MALDI).

¹H-NMR (500 MHz, CD₂Cl₂): δ 8.08 (d, 4H), 7.84 (t, 1H), 7.74 (t, 2H), 7.69 (d, 2H), 7.41 (d, 4H), 2.00 (m, 3H), 1.89 (m, 6H), 1.71 (m, 6H), HRMS(MALDI) calculated for C₃₅H₂₈F₄I₂O₅S₂: m/z 921.94 Found: 921.95.

### Synthesis Example 2: Synthesis of Compound PAG B

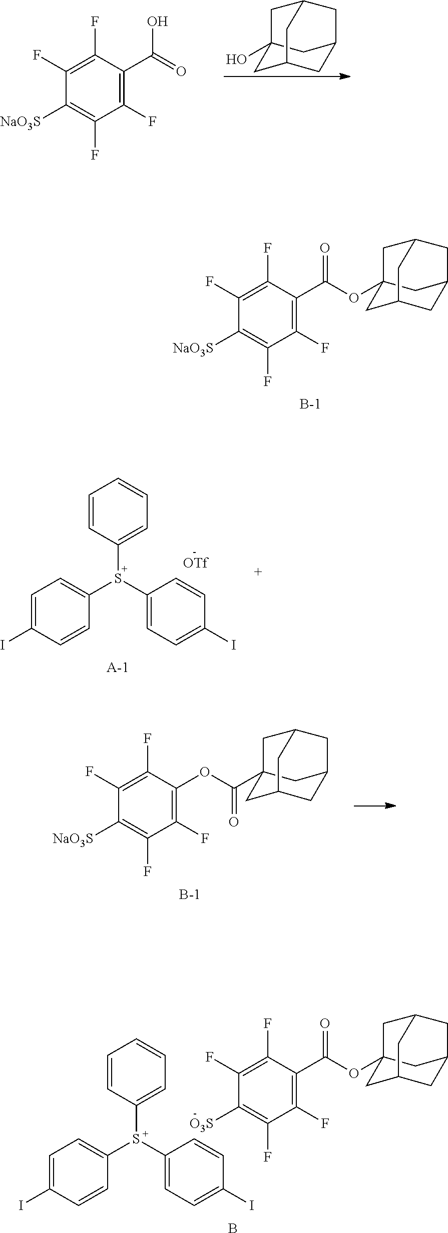

### Synthesis of Compound B-1

Sodium 4-carboxy-2,3,5,6-tetrafluorobenzenesulfonate) (1.490 g, 5.03 mmol) and adamantan-1-ol (0.766 g, 5.03 mmol) were dissolved in 50 ml of dichloromethane and 1 ml of dimethylformamide, and then dicyclohexylcarbodiimide (1.141 g, 5.53 mmol), 4-dimethylaminopyridine) (0.673 g, 5.53 mmol), and trimethylamine (0.78 ml, 5.53 mmol) were added thereto, and a reaction was allowed to occur therebetween at room temperature for 16 hours. After the reaction, the precipitate was removed by filtering and washed twice with brine in a separatory funnel, and the organic layer was column purified under a condition of DCM:MeOH=20:1 (volume ratio) to thereby obtain compound B-1 (1.06 g, yield: 49%). The produced compound was identified by NMR and LC-MS.

¹H-NMR (500 MHz, CD₂Cl₂): δ 2.17 (m, 6H), 2.00 (m, 3H), 1.71 (m, 6H), LC-MS m/z=407.06 (anion).

### Synthesis of Compound PAG B

Compound PAG B (yield: 48.2%) was obtained using the same method as that used for the synthesis of Compound PAG A of Synthesis Example 1, except that Compound B-1 was used instead of Compound A-2. The produced compound was identified by NMR and MALDI.

¹H-NMR (500 MHz, CD₂Cl₂): δ 8.08 (d, 4H), 7.84 (t, 1H), 7.74 (t, 2H), 7.69 (d, 2H), 7.41 (d, 4H), 2.17 (m, 6H), 2.00 (m, 3H), 1.71 (m, 6H), HRMS(MALDI) calculated for C₃₅H₂₈F₄I₂O₅S₂: m/z 921.94 Found: 921.94.

### Synthesis Example 3: Synthesis of Compound PAG C

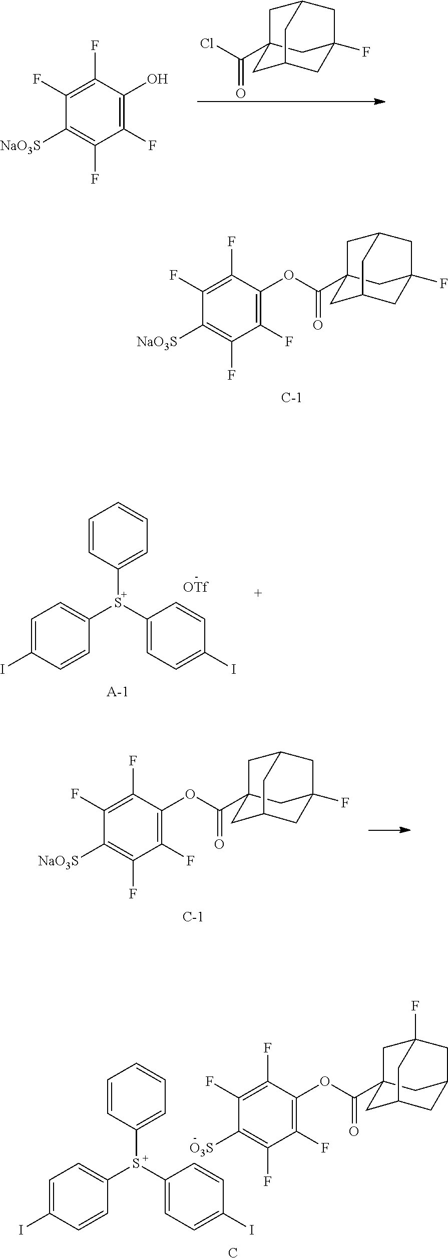

### Synthesis of Compound C-1

Compound C-1 (yield: 75%) was obtained using the same method as that used for the synthesis of Compound A-2 of Synthesis Example 1, except that 3-fluoroadamantane-1-carbonyl chloride was used instead of 1-adamantanecarbonyl chloride. The produced compound was identified by NMR and LC-MS.

¹H-NMR (500 MHz, CD₂Cl₂): δ 1.98 (m, 2H), 1.61 (m, 9H), 1.18 (m, 3H), LC-MS m/z=448.04 (anion).

### Synthesis of Compound PAG C

Compound PAG C (yield: 46.7%) was obtained using the same method as that used for the synthesis of Compound PAG A of Synthesis Example 1, except that Compound C-1 was used instead of Compound A-2. The produced compound was identified by NMR and MALDI.

¹H-NMR (500 MHz, CD₂Cl₂): δ 8.08 (d, 4H), 7.84 (t, 1H), 7.74 (t, 2H), 7.69 (d, 2H), 7.41 (d, 4H), 1.98 (m, 2H), 1.61 (m, 9H), 1.18 (m, 3H), HRMS(MALDI) calculated for C₃₅H₂₇F₅I₂O₅S₂: m/z 939.93 Found: 939.94.

### Synthesis Example 4: Synthesis of Compound PAG D

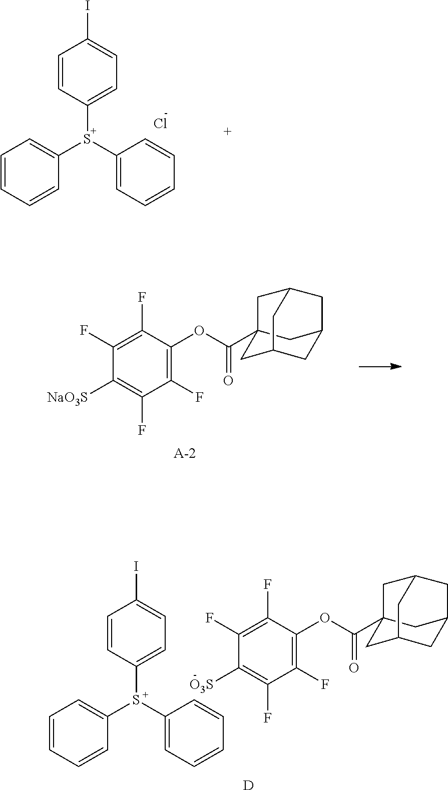

### Synthesis of Compound PAG D

Compound PAG D (yield: 42.4%) was obtained using the same method as that used for the synthesis of Compound PAG A of Synthesis Example 1, except that (4-iodophenyl)diphenylsulfonium triflate was used instead of Compound A-1. The produced compound was identified by NMR and MALDI.

¹H-NMR (500 MHz, CD₂Cl₂): δ 8.08 (d, 2H), 7.75 (m, 10H), 7.41 (d, 2H), 7.69 (d, 2H), 7.41 (d, 4H), 2.00 (m, 3H), 1.89 (m, 6H), 1.71 (m, 6H), HRMS(MALDI) calculated for C₃₅H₂₉F₄IO₅S₂: m/z 796.04 Found: 796.04.

### Synthesis Example 5: Synthesis of Polymer X

Polymer X was synthesized with reference to KR 2022-0074627 A. Specifically, 0.94 g of dimethyl 2,2′-azobis(2-methylpropionate) (Waco Chemicals), 3.03 g of 2-ethyl-2-adamantyl methacrylate (TCI Chemicals), and 1.98 g of 4-acetoxy styrene (Sigma-Aldrich) were dissolved in tetrahydrofuran, followed by polymerization at 80° C. for 8 hours, to thereby obtain Polymer X.

Polymer X has the following structure, wherein x and y are each 50.

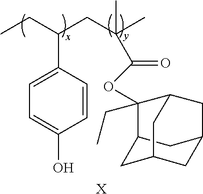

### Evaluation Example 1: Evaluation of Acid Generating Effect

The acid generating effect was examined in the following manner. 10 wt % of poly(4-vinylphenol) (Mw: about 11,000, available from Sigma-Aldrich) and 6.5 wt % of Coumarin 6 (CAS No. 38215-36-0) were dissolved in cyclohexanone, and the photoacid generators shown in Table 1 were added thereto along with Coumarin 6 in the same number of moles, respectively. The resulting solution was spin-coated on a 1 inch×1 inch quartz plate to a thickness of 400 nm and dried at 130° C. for 2 minutes to thereby form a thin film. The thin film was exposed to DUV rays (248 nm) at a dose of 10 mJ/cm² to 100 mJ/cm², and then absorbance was measured. Theoretically, since the absorbance of coumarin 6 increases at a wavelength of 535 nm by acid generation, coumarin 6 was used as an acid indicator. After assuming that coumarin 6 was 100% converted for the absorbance intensity after 100 mJ/cm² exposure, the absorbance intensity at each exposure dose was normalized to indicate the degree of acid generation.

TABLE 1

| | | Degree of acid |
|---|---|---|
| | | generation |
| | Photoacid generator | @ DUV 20 mJ |
| Example 1 | A | 106.4 |
| Example 2 | B | 105.9 |
| Example 3 | C | 106.2 |
| Example 4 | D | 103.7 |
| Comparative | 1 | 100 |
| Example 1 | | |
| Comparative | 2 | 97.4 |
| Example 2 | | |
| Comparative | 3 | 100.2 |
| Example 3 | | |
| Comparative | 4 | 101.6 |
| Example 4 | | |

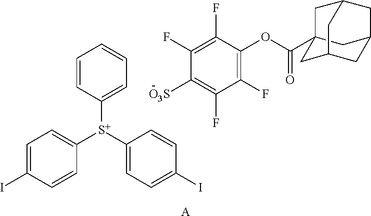

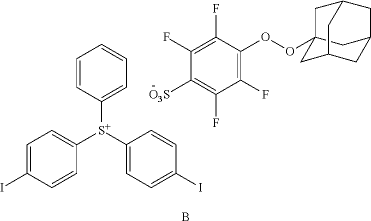

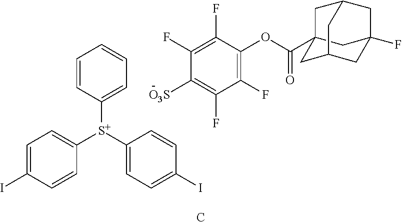

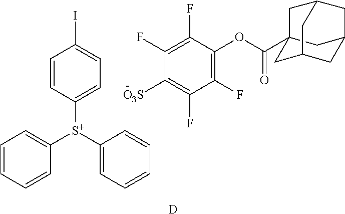

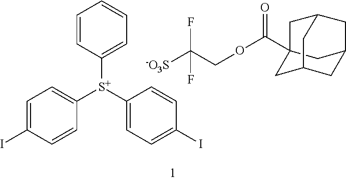

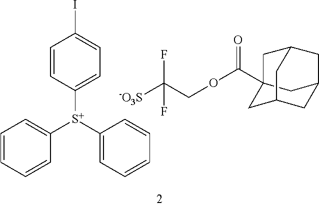

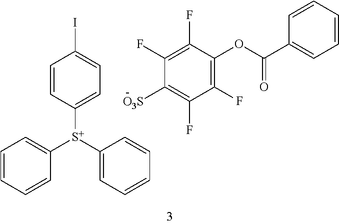

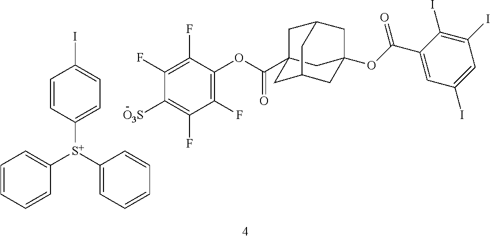

Referring to Table 1, it can be confirmed that Examples 1 to 4 have an improved acid generating effect compared to Comparative Examples 1 to 4. That is, it can be predicted that, when Examples 1 to 4 are used as photoacid generators, a more improved resolution can be provided even when the same amount of the photoacid generator is used.

### Evaluation Example 2: Evaluation of Solubility (Dark-Loss) by Etchant

Polymer X was dissolved in a 7/3 (wt/wt) solution of propylene glycol methyl ether/propylene glycol methyl ether acetate (PGME/PGMEA), and the photoacid generators shown in Table 2 were respectively added thereto. The resulting solution was spin-coated on a silicon wafer at 1500 rpm for 60 seconds. A coating layer having a thickness of 100 nm was formed by prebaking at 110° C. for 60 seconds. Thereafter, the coating layer was dipped in an aqueous 2.38 wt % TMAH solution for 60 seconds and cleaned with deionized (DI) water, and then the thickness of the remaining coating layer was measured using a three-dimensional optical profiler (Bruker, Contour X-100), and changes in the thickness of the coating layer were normalized (shown as a relative value based on Comparative Example 1), and the results thereof are shown in Table 2.

TABLE 2

| | Photoacid generator | Dark-loss |
|---|---|---|
| Example 1 | A | 49.1 |
| Example 2 | B | 50.2 |
| Example 3 | C | 51.5 |
| Example 4 | D | 51.7 |
| Comparative | 1 | 100 |
| Example 1 | | |
| Comparative | 2 | 145 |
| Example 2 | | |
| Comparative | 3 | 52 |
| Example 3 | | |
| Comparative | 4 | 59 |
| Example 4 | | |

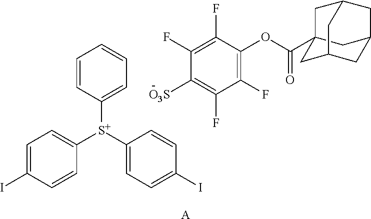

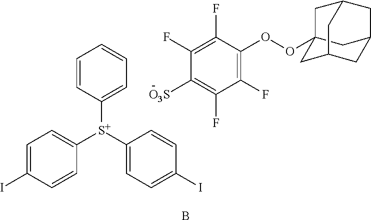

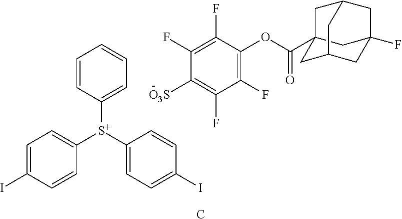

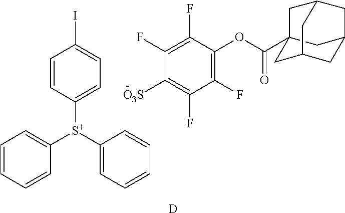

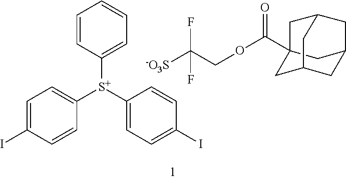

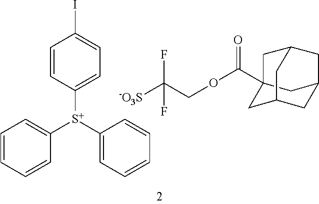

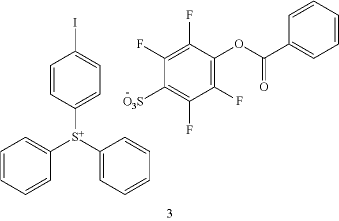

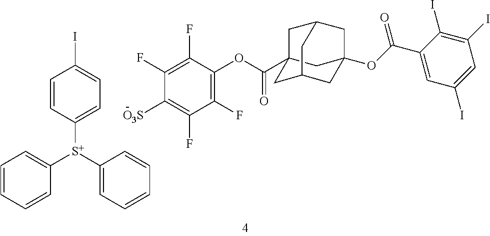

Referring to Table 2, it can be confirmed that the solubility of the non-exposed region (dark-loss) of each of Examples 1 to 4 in an etchant is lower than that of Comparative Examples 1 to 4. That is, it can be seen that, when the photoacid generators of Examples 1 to 4 are used, patterns with more improved resolution can be provided.

### Evaluation Example 3. Photoresist Performance Evaluation

A 12-inch circular silicon wafer substrate was pretreated for 10 minutes under a UV ozone cleaning system. Polymer X was dissolved in a 7/3 (wt/wt) solution of propylene glycol methyl ether/propylene glycol methyl ether acetate (PGME/PGMEA), and the photoacid generators shown in Table 3 were added thereto respectively. The resulting solution was spin-coated on a silicon wafer at 1500 rpm for 60 seconds. A coating layer with a thickness of 100 nm was formed by prebaking at 110° C. for 60 seconds. Then, EUV (ASML NXE-3350) rays were projected onto a mask having a C/H pattern with a CD size of 25 nm and a pitch of 54 nm. Then, a post-exposure bake was performed at 90° C. for 60 seconds, followed by immersion in an aqueous 2.38 wt % TMAH solution for 60 seconds, followed by cleansing with deionized (DI) water for 10 seconds to remove the coated portion not exposed to EUV light and dry, to thereby form a resist pattern. The Eₒₚ, resolution, LWR, and sensitivity of the photoresist pattern were measured using a critical dimension measurement scanning electron microscope (CD-SEM). The Z-factor was calculated by substituting the measured value into Equation 1 below, and the results thereof were normalized and are shown in Table 3 below.

$$Z - factor = {( Resolution )}^{3} \times {( LWR )}^{2} \times ( Sensitivity ) \qquad Equation 1$$

In Equation 1, resolution is a CD size (half pic), LWR is a line width roughness, sensitivity is Eₒₚ (dose), and the lower the Z-factor, the better the pattern performance at the same dose.

TABLE 3

| | Photoacid generator | Z-factor |
|---|---|---|
| Example 1 | A | 97.3 |
| Example 2 | B | 98.6 |
| Example 3 | C | 97.1 |
| Example 4 | D | 99.4 |
| Comparative | 1 | 100 |
| Example 1 | | |
| Comparative | 2 | 103.2 |
| Example 2 | | |
| Comparative | 3 | 105.7 |
| Example 3 | | |
| Comparative | 4 | 107.2 |
| Example 4 | | |

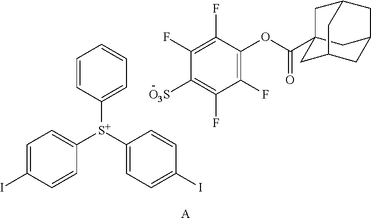

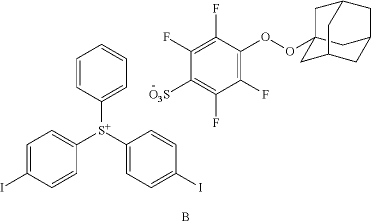

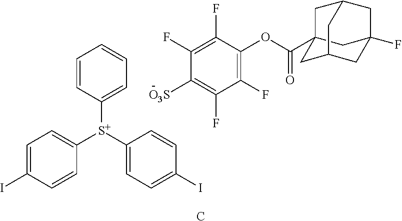

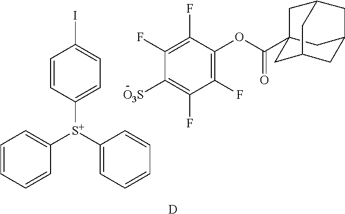

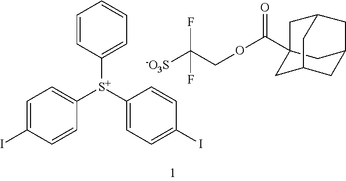

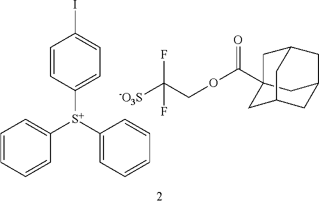

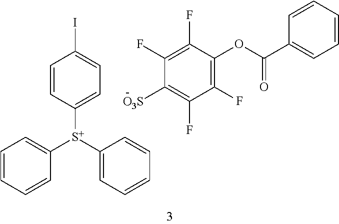

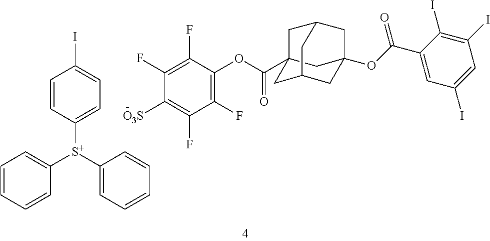

Referring to Table 3, it can be confirmed that the photoresist patterns to which the photoresist films formed of the photoresist compositions of Examples 1 to 4 are applied have lower Z-factors than the photoresist patterns to which the photoresist films formed of the photoresist compositions of Comparative Examples 1 to 4.

Embodiments of the disclosure may provide: an organic salt capable of acting as a photoacid generator that can provide improved sensitivity and/or resolution; and a resist composition including the organic salt.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An organic salt represented by Formula 1:
A₁₁_{⁺}B₁₁_{^{−}} Formula 1
wherein, in Formula 1,
A₁₁_{⁺} is represented by Formula 1A and B₁₁_{^{−}} is represented by Formula 1B, 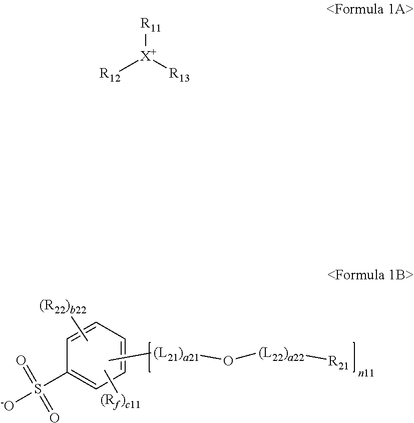
wherein, in Formulae 1A and 1B,
X is sulfur (S), selenium (Se), or tellurium (Te);
R₁₁ to R₁₃ are each independently a linear, branched, or cyclic C₁-C₃₀ monovalent hydrocarbon group optionally containing a heteroatom;
at least one of R₁₁ to R₁₃ contains at least one iodine (1);
adjacent two of R₁₁ to R₁₃ are optionally bonded to each other to form a condensed ring;
L₂₁ and L₂₂ are each independently a single bond or a divalent linking group;
a21 and a22 are each independently an integer from 1 to 3;
R₂₁ is a C₁-C₃₀ cycloalkyl group optionally containing deuterium, a halogen, a hydroxyl group, a cyano group, a carbonyl group, a carboxyl group, an ether bond, an ester bond, a sulfonate ester bond, a carbonate, a lactone ring, a sultone ring, a carboxylic anhydride moiety, a haloalkyl moiety, or any combination thereof;
R₂₂ is hydrogen; deuterium; a halogen; or a linear, branched, or cyclic C₁-C₃₀ monovalent hydrocarbon group optionally containing a heteroatom;
R_{f} is fluorine (F) or a C₁-C₃₀ alkyl group substituted with F;
b22 is an integer from 0 to 3;
c11 is an integer from 1 to 4; and
n11 is an integer from 1 to 4.

2. The organic salt of claim 1, wherein X is S.

3. The organic salt of claim 1, wherein
R₁₁ to R₁₃ are each independently selected from a C₁-C₂₀ alkyl group, a C₃-C₂₀ cycloalkyl group, and a C₆-C₂₀ aryl group, each unsubstituted or substituted with deuterium, a halogen, a cyano group, a hydroxyl group, a carboxyl group, an ester moiety, a sulfonate ester moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

4. The organic salt of claim 1, wherein A₁₁_{⁺} contains 1 I atom, 2 I atoms, or 3 I atoms.

5. The organic salt of claim 1, wherein A₁₁_{⁺} is represented by Formula 1A-1: 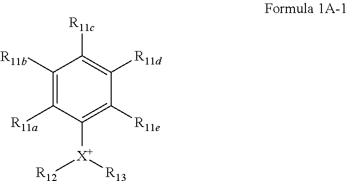
wherein, in Formula 1A-1,
X is S, Se, or Te;
R₁₁ₐ to R₁₁ₑ are each independently: hydrogen; a halogen; or a linear, branched, or cyclic C₁-C₂₀ monovalent hydrocarbon group optionally containing a heteroatom;
R₁₂ and R₁₃ are each independently a linear, branched, or cyclic C₁-C₃₀ monovalent hydrocarbon group optionally containing a heteroatom;
an adjacent two of R₁₁ₐ to R₁₁ₑ, R₁₂, and R₁₃ are optionally bonded to each other to form a condensed ring; and
at least one of R₁₁ₐ to R₁₁ₑ, R₁₂, and R₁₃ contains at least one I atom.

6. The organic salt of claim 1, wherein A₁₁_{⁺} is represented by Formula 1A-11 or 1A-12: 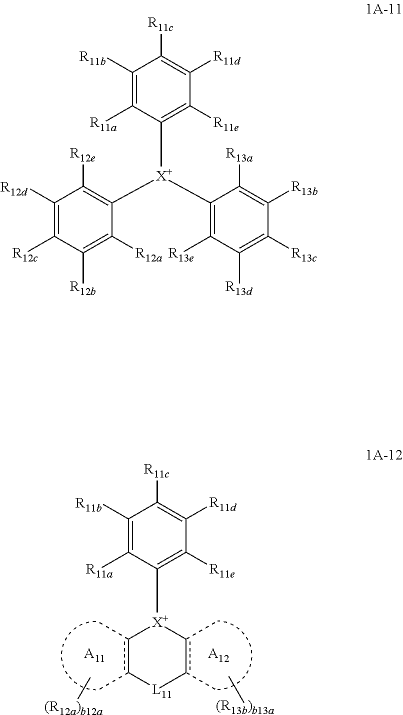
wherein, in Formulae 1A-11 and 1A-12,
X is S, Se, or Te;
R₁₁ₐ to R₁₁ₑ, R₁₂ₐ to R₁₂ₑ, and R₁₃ₐ to R₁₃ₑ are each independently: hydrogen;
a halogen; or a linear, branched, or cyclic C₁-C₂₀ monovalent hydrocarbon group optionally containing a heteroatom;
at least one of R₁₁ₐ to R₁₁ₑ, R₁₂ₐ to R₁₂ₑ, and R₁₃ₐ to R₁₃ₑ is I, or at least one of R₁₁ₐ to R₁₁ₑ, R₁₂ₐ, and R_{13b} is I;
b12a and b13a are each an integer from 1 to 4;
each of A11 and A12 do not exist or is a benzene ring; [symbol]
is a carbon-carbon single bond or a carbon-carbon double bond;
L₁₁ is a single bond, O, S, CO, SO, SO₂, CRR′, or NR;
R and R′ are each independently hydrogen; deuterium; a halogen; a cyano group; a hydroxyl group; or a linear, branched, or cyclic C₁-C₂₀ monovalent hydrocarbon group optionally containing a heteroatom; and
an adjacent two of R₁₁ₐ to R₁₁ₑ, R₁₂ₐ to R₁₂ₑ, and R₁₃ₐ to R₁₃ₑ are optionally bonded to each other to form a condensed ring.

7. The organic salt of claim 1, wherein A₁₁_{⁺} is selected from Group I: 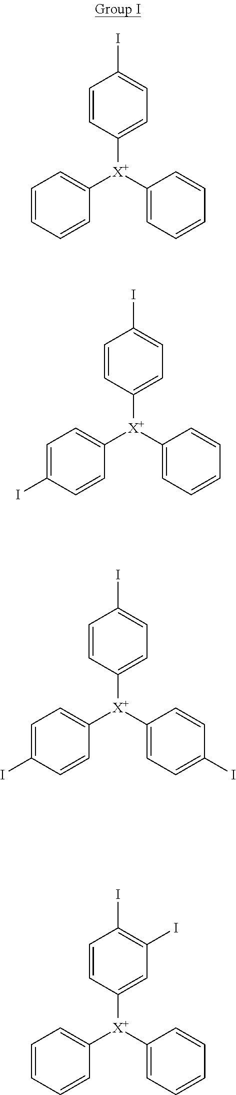 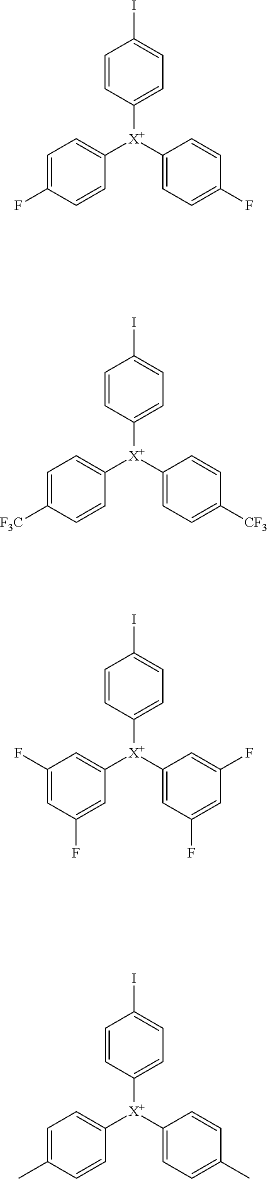 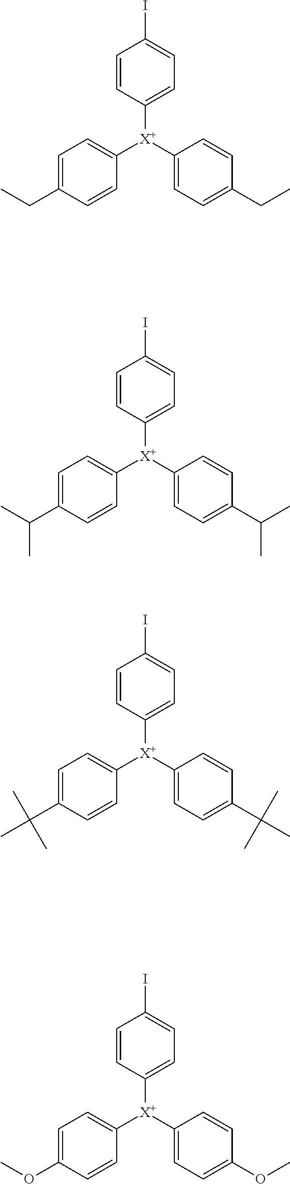 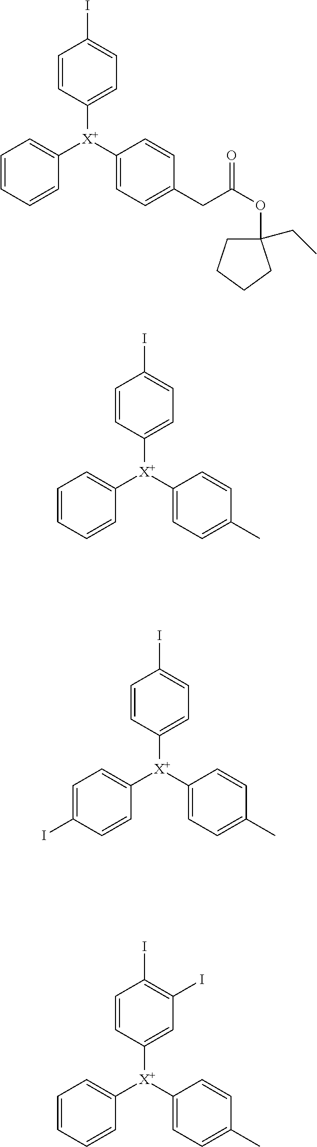 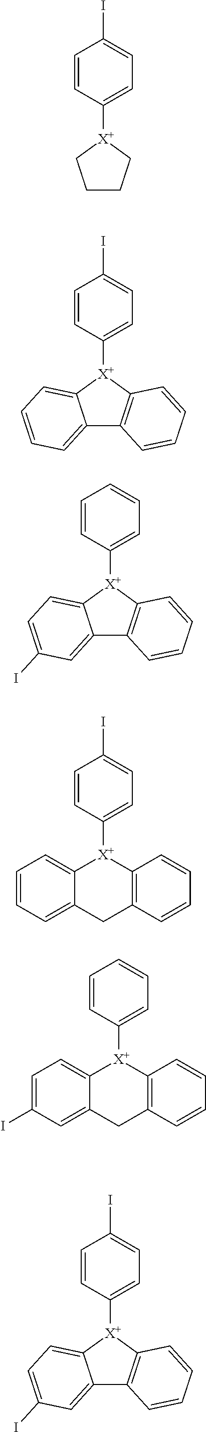 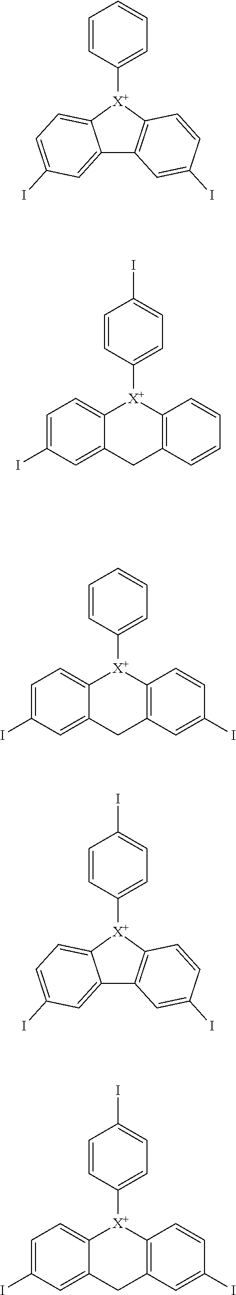 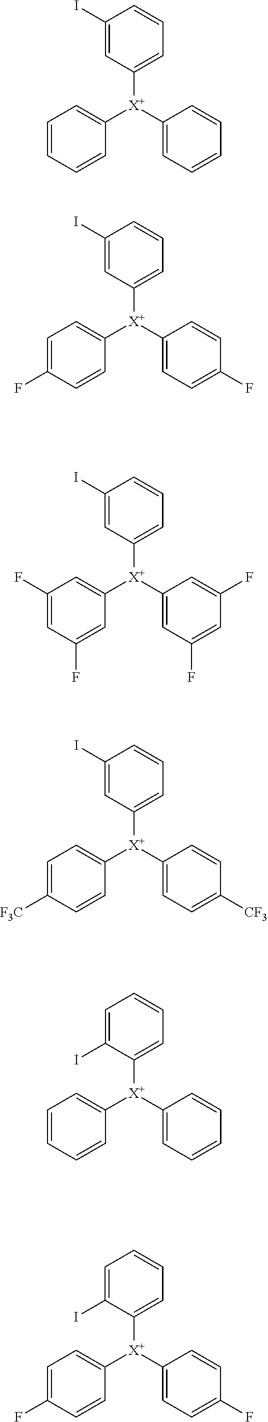 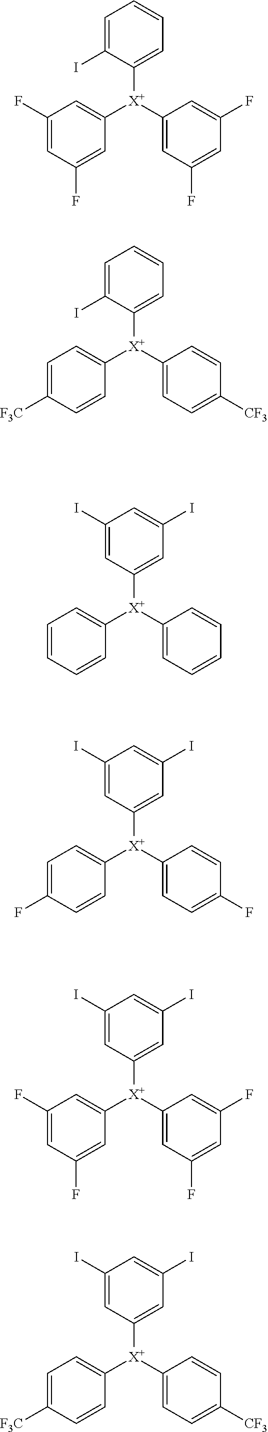
wherein, in Group I,
X may be S, Se, or Te.

8. The organic salt of claim 1, wherein L₂₁ and L₂₂ are each independently a single bond, O, C(═O), C(═O)O, OC(═O), C(═O)NH, NHC(═O), or a linear, branched, or cyclic divalent hydrocarbon group optionally containing a heteroatom.

9. The organic salt of claim 1, wherein
R₂₁ selected from a cyclopentyl group, a cyclohexyl group, a cycloheptyl group, a cyclooctyl group, a bicyclo[3.1.0]hexyl group, a bicyclo[3.2.0]heptyl group, a bicyclo[3.3.0]octyl group, a bicyclo[4.3.0]nonyl group, a bicyclo[4.4.0]decanyl group, a bicyclo[1.1.1]pentyl group, a bicyclo[2.1.1]hexyl group, a bicyclo[2.2.1]heptyl group, a bicyclo[2.2.2]octyl group, an adamantanyl group, a tricyclo[5.2.1.02,6]decanyl group, and a tetracyclo[6.2.1.13,6.02,7]dodecanyl group, each optionally containing deuterium, a halogen, a hydroxyl group, a carbonyl group, a carboxyl group, an ether bond, an ester bond, a carbonate, a lactone ring, a carboxylic anhydride moiety, a haloalkyl moiety, or any combination thereof.

10. The organic salt of claim 1, wherein
R₂₂ is selected from hydrogen; deuterium; a halogen; a C₁-C₂₀ alkyl group, a C₃-C₂₀ cycloalkyl group, and a C₆-C₂₀ aryl group, each being unsubstituted or substituted with deuterium, a halogen, a cyano group, a hydroxyl group, a carboxyl group, an ester moiety, a sulfonate ester moiety, a carbonate moiety, a lactone moiety, a sultone moiety, a carboxylic anhydride moiety, a C₁-C₂₀ alkyl group, a C₁-C₃₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

11. The organic salt of claim 1, wherein
R_{f} is one of F, CF₃, CF₂H, CFH₂, CH₂CF₃, CH₂CF₂H, CH₂CFH₂, CHFCH₃, CHFCF₂H, CHFCFH₂, CHFCF₃, CF₂CF₃, CF₂CF₂H, or CF₂CFH₂, or
R_{f} is represented by one Formulae 10-1 to 10-14, 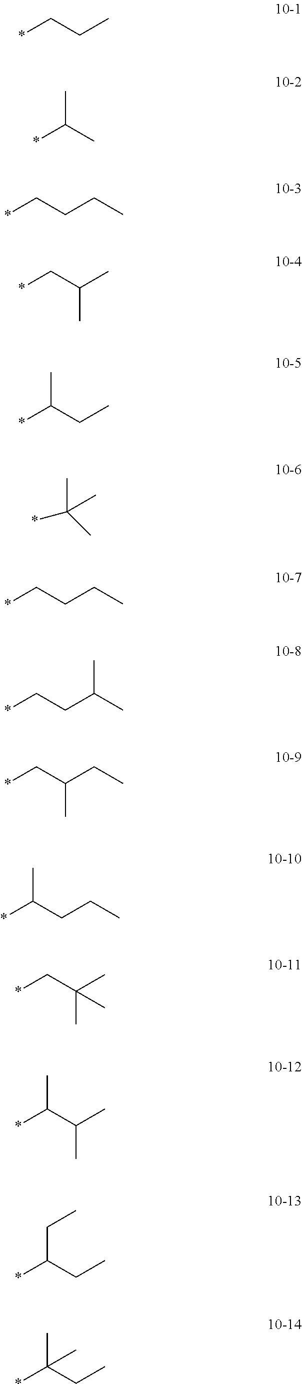
wherein, in Formulae 10-1 and 10-14,
at least one hydrogen is substituted with —F,
* indicates a binding site to a neighboring atom.

12. The organic salt of claim 1, wherein B₁₁_{^{−}} is represented by Formula 1B-1: 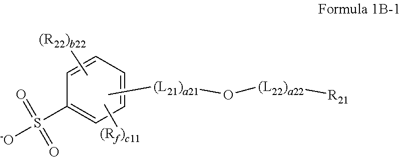
wherein, in Formula 1B-1,
L₂₁, L₂₂, a21, a22, R₂₁, R₂₂, R_{f}, b22, and c11 are as defined in connection with Formula 1B.

13. The organic salt of claim 1, wherein B₁₁_{^{−}} is represented by any one of Formulae 1B-11 and 1B-12: 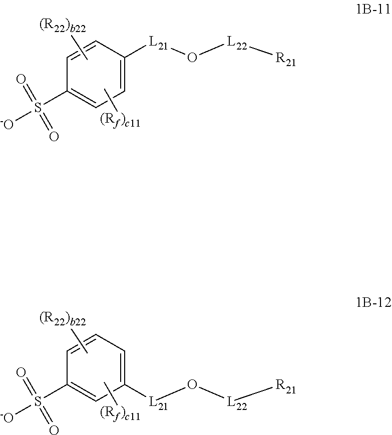
wherein, in Formulae 1B-11 and 1B-12,
L₂₁, L₂₂, a21, a22, R₂₁, R₂₂, R_{f}, b22, and c11 are as defined in connection with Formula 1B.

14. The organic salt of claim 1, wherein B₁₁_{^{−}} is selected from Group II:

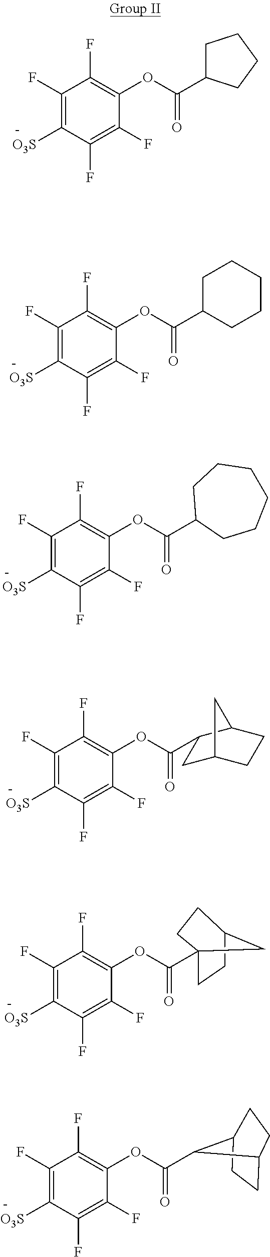

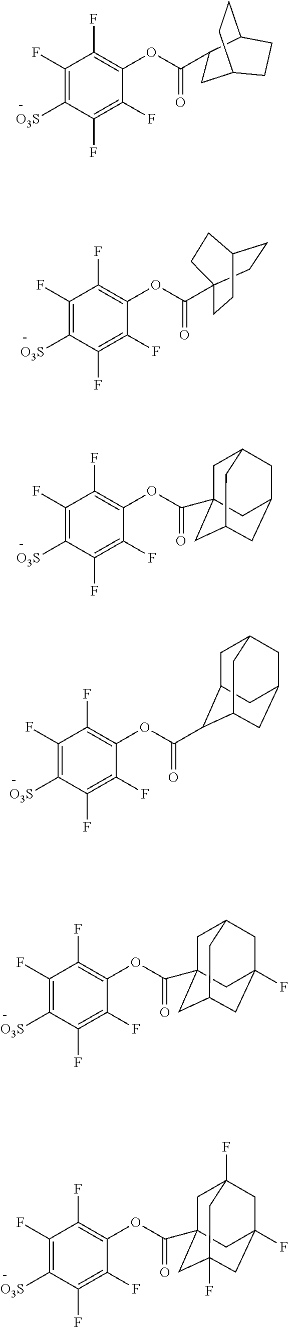

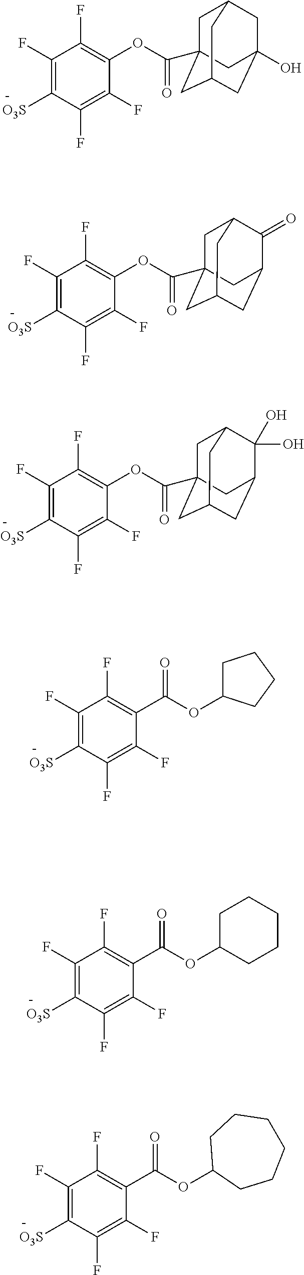

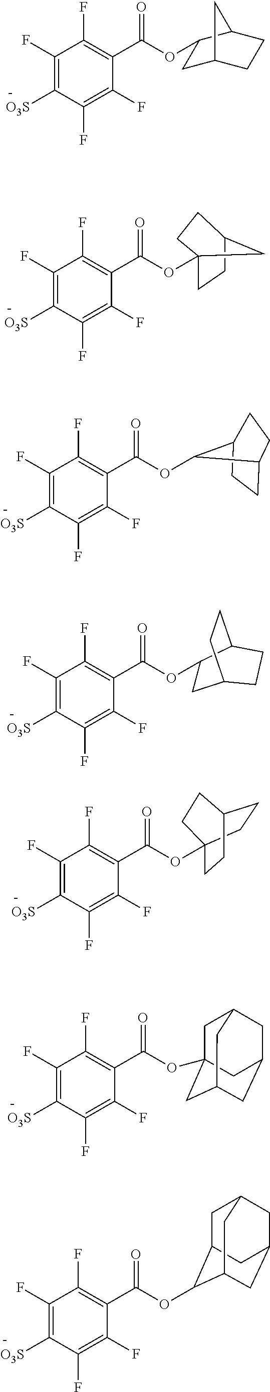

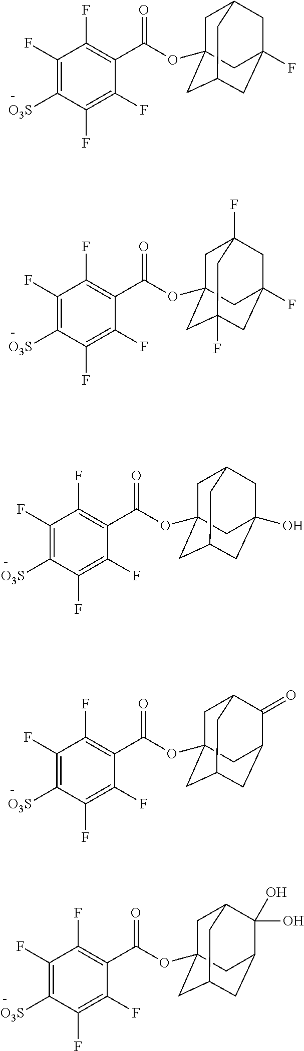

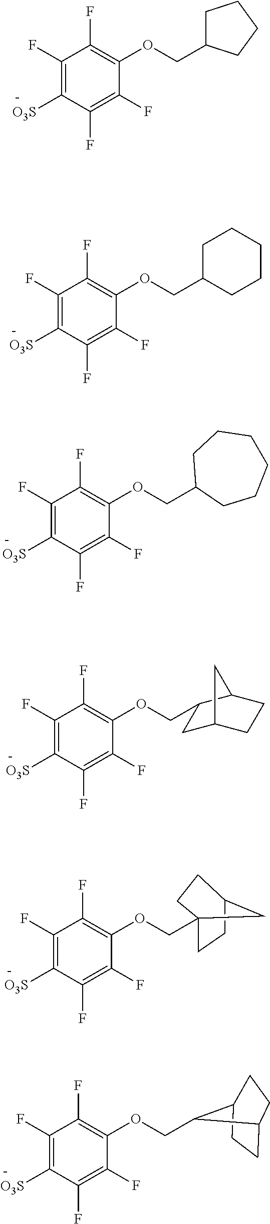

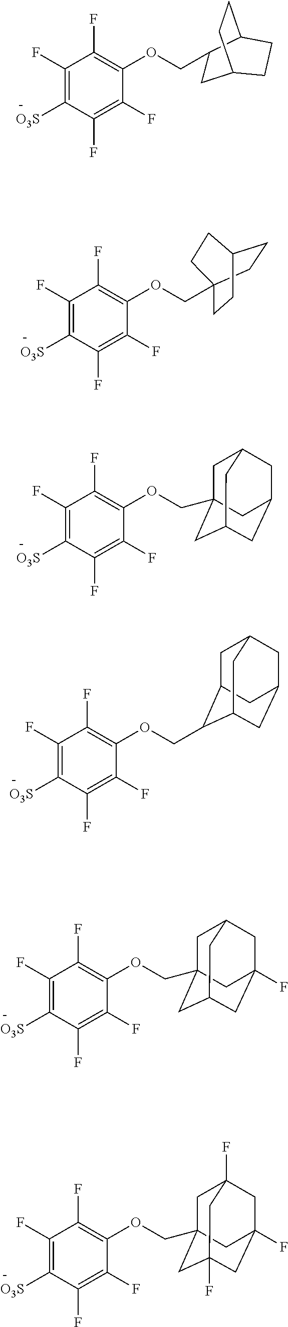

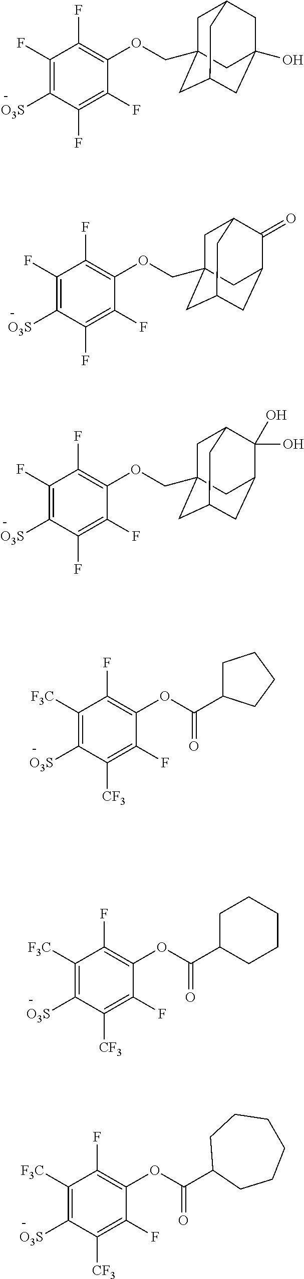

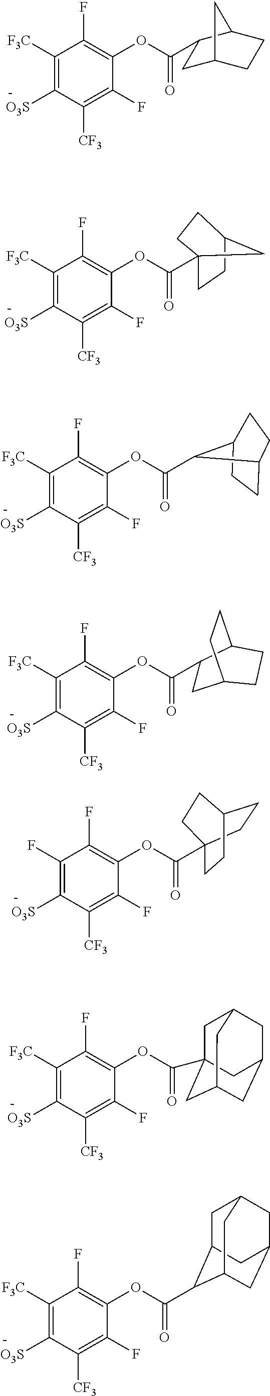

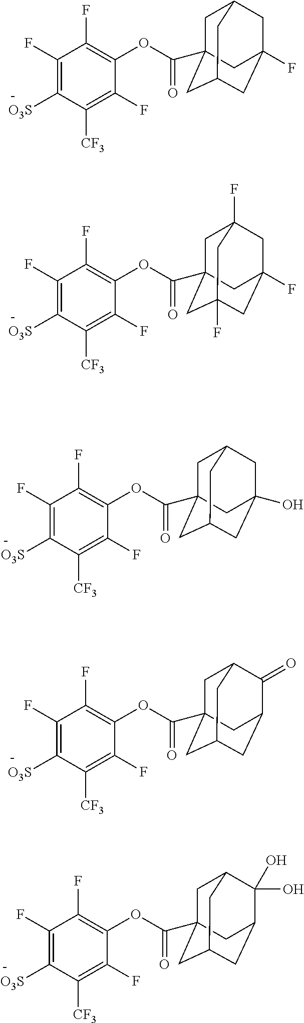

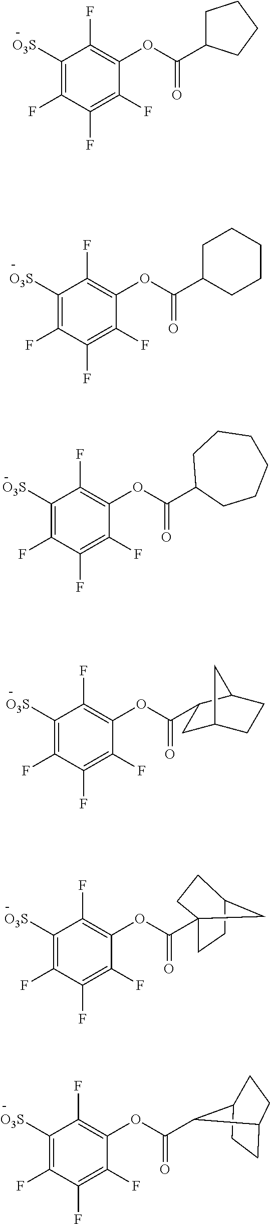

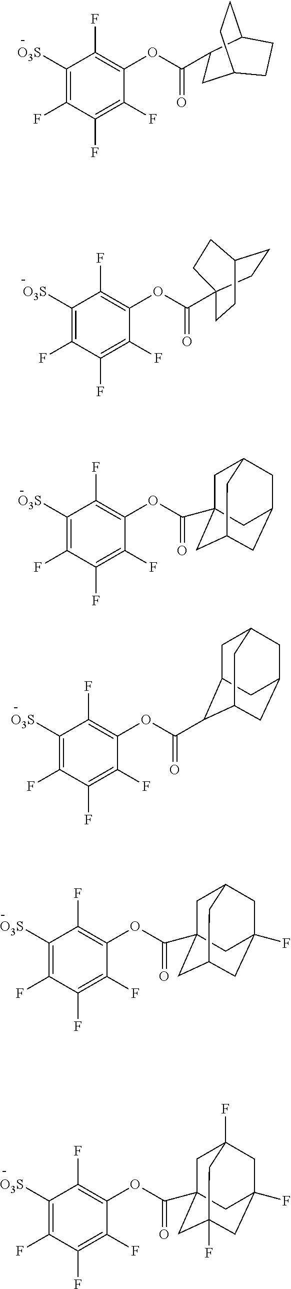

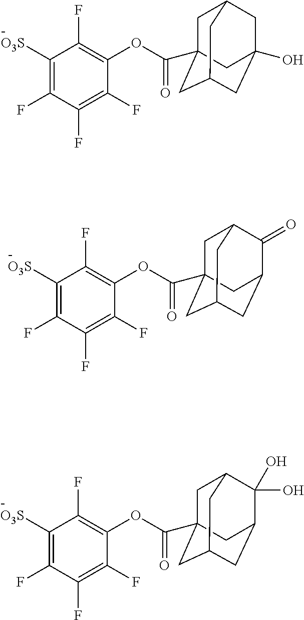

15. A resist composition comprising:
the organic salt of claim 1;
an organic solvent; and
a base resin.

16. The resist composition of claim 15, wherein the organic salt is a photodegradable compound that is configured to generate an acid upon exposure to light.

17. The resist composition of claim 15, further comprising:
a quencher.

18. The resist composition of claim 15, wherein
an amount of the organic salt is in a range of about 0.1 parts by weight to about 40 parts by weight with respect to 100 parts by weight of the base resin.

19. A method of forming a pattern, comprising:
forming a resist film by applying the resist composition of claim 15 onto a substrate;
exposing at least a portion of the resist film to high-energy rays; and
developing the exposed resist film by using a developer.

20. The method of claim 19, wherein the exposing is performed by irradiating ultraviolet (UV) rays, deep ultraviolet (DUV) rays, extreme ultraviolet (EUV) rays, and/or electron beams (EB).
